(19) United States
(12) Patent Application Publication
(10) Pub. No.: US 2025/0332223 A1
(43) Pub. Date: Oct. 30, 2025

(54) GHRH ANTAGONISTS FOR USE IN A METHOD OF TREATING SARCOIDOSIS

(71) Applicants: University of Miami, Miami, FL (US); The United States Government as Represented by the Department of Veterans Affairs, Washington, DC (US)

(72) Inventors: Mehdi Mirsaeidi, Jacksonville, FL (US); Chongxu Zhang, Riverside, IL (US); Andrew V. Schally, Miami Beach, FL (US); Renzhi Cai, Fulshear, TX (US)

(21) Appl. No.: 19/255,898

(22) Filed: Jun. 30, 2025

Related U.S. Application Data
(63) Continuation of application No. 17/648,137, filed on Jan. 17, 2022, now Pat. No. 12,377,135.
Application No. 17/648,137 is a continuation of application No. PCT/US2020/042540, filed on Jul. 17, 2020.
(60) Provisional application No. 62/875,703, filed on Jul. 18, 2019.

(51) Int. Cl. A61K 38/25; A61P 11/00

(52) U.S. Cl. CPC A61K 38/25; A61P 11/00

(57) ABSTRACT

The disclosure provides a method of treating sarcoidosis, the method comprising administering a GHRH antagonist to mammalian subject in need thereof.

### CROSS REFERENCE TO RELATED APPLICATIONS

The instant application is a continuation of U.S. patent application Ser. No. 17/648,137 filed on Jan. 17, 2022, which is a continuation application under 35 U.S.C. 111(a) of international patent application number PCT/US2020/042540 filed on Jul. 17, 2020 and designating the United States, which claimed the priority of U.S. provisional patent application 62/875,703, filed on Jul. 18, 2019, which are incorporated by reference in their entirety.

### STATEMENT AS TO FEDERALLY SPONSORED RESEARCH

This invention was made with government support under a Distinguished Scientist grant awarded by the Department of Veterans Affairs and under grant number P30CA240139 awarded by the National Institutes of Health (NIH). The government has certain rights in the invention.

### INCORPORATION OF THE SEQUENCE LISTING

The present application contains a sequence listing that is submitted concurrently with the filing of this application, containing the file name “7085-0020_SL.xml” Size: 51,954 bytes, created on May 22, 2025, and is herein incorporated by reference in its entirety.

### FIELD OF THE INVENTION

The invention relates to materials and methods for treating sarcoidosis.

### BACKGROUND

Sarcoidosis is a multi-organ granulomatous disease of unknown etiology that is associated with significant morbidity and mortality in the US and affects hundreds of thousands of people around the world. Mirsaeidi et al., Chest. 2015; 147(2):438-449. Although the etiology of this condition is not well-known, there are significant similarities between sarcoidosis and other granuloma-forming disorders including mycobacterial and other microbial infections and environmental agent-induced granuloma. Chen et al., Clinics in chest medicine. 2008; 29(3):365-377, vii. In the affected organ, sarcoidosis triggers an early inflammatory reaction characterized by cellular recruitment of TH1 helper cells, followed by a later phase where macrophage recruitment leads to granuloma formation. In certain patients, anti-inflammatory responses, including cytokines and apoptosis, are activated to facilitate tissue healing and repair. Koh et al., Expert Rev Mol Med. 2011; 13:e23. Almost 50% of sarcoidosis patients require systemic steroid therapy. In up to 20% of patients, the inflammatory process continues despite steroids and leads to tissue remodeling with fibrosis (permanent scarring of affected tissue). Patterson et al., Annals of the American Thoracic Society. 2013; 10(4):362-370.

Given the multiorgan involvement of sarcoidosis in more than 50% of patients, the treatment of this disease is challenging. Corticosteroid is the cornerstone of therapy, and the US Food and Drug Administration (FDA) has approved only two medications (prednisone and Acthar-Gel) for sarcoidosis 6, 7. Miller et al., Ann Intern Med. 1952; 37(4):776-784; Baughman et al., Respir Med. 2016; 110:66-72. However, these agents cause significant side effects after prolonged use, making them undesirable for long-term treatment. In patients with persistent symptoms and complicated presentation with involvement in vital organs, treatment should be started immediately and be continued for months, thus signaling the need of an alternative strategy which is less toxic and tolerable.

### SUMMARY

1. The disclosure provides a method of treating sarcoidosis, the method comprising administering a GHRH antagonist to mammalian subject in need thereof. The disclosure further provides use of a GHRH antagonist for treating sarcoidosis or in the preparation of a medicament for treating sarcoidosis. The disclosure also provides a GHRH antagonist for use in treating sarcoidosis.

2. In various aspects, such as the method or use of paragraph 1, the GHRH antagonist comprises the amino acid sequence (Formula I): R¹-Tyr¹-D-Arg²-Asp³-A⁴-Ile⁵-A⁶-Thr⁷-A⁸-Har⁹-A¹⁰-A¹¹-A¹²-Val¹³-Leu¹⁴-A¹⁵-Gln¹⁶-A¹⁷-Ser¹⁸-Ala¹⁹-A²⁰-A²¹-Leu²²-Leu²³-Gln²⁴-Asp²⁵-Ile²⁶-Nle²⁷-D-Arg²⁸-A²⁹-R²-R³-NH₂ (SEQ ID NO: 2),
wherein R¹ is PhAc (phenylacetyl), Nac (naphthylacetyl), Oct (octanoyl), N-Me-Aib (N-methyl-alpha-aminoisobutyroyl), Dca (dichloroacetyl), Ac-Ada (acetyl-12-aminododecanoyl), Fer (ferulyl), Ac-Amc (acetyl-8-aminocaprylyl), Me-NH-Sub (methyl-NH-suberyl), PhAc-Ada (phenylacetyl 12-aminododecanoyl), Ac-Ada-D-Phe, Ac-Ada-Phe, Dca-Ada (dichloroacetyl-12-aminododecanoyl), Nac (naphthylacetyl), Nac-Ada, Ada-Ada, or CH₃(CH₂)₁₀—CO-Ada;
A⁴ is Ala or Me-Ala;
A⁶ is Cpa (para-chlorophenylalanine) or Phe(F)5 (pentafluoro-phenylalanine, also referred to as Fpa5);
A⁸ is Ala, Pal (pyridylalanine), Dip ((3,3-diphenyl)alanine), or Me-Ala;
A¹⁰ is Fpa5, Tyr(Alk) where Alk is Me or Et;
A¹¹ is His or Arg;
A¹² is Lys, Lys(0-11) (SEQ ID NO: 9) (Lys(A0-A1-A2-A3-A4-A5-A6-A7-A8-A9-A10-A11-), Lys(Me)₂, or Orn (ornithine);
A¹⁵ is Abu (alpha-aminobutyric acid) or Orn;
A¹⁷ is Leu or Glu;
A²⁰ is Har (homoarginine) or His;
A²¹ is Lys, Lys(Me)₂ or Orn;
A²⁹ is Har, Arg or Agm (agmatine);
R² is β-Ala, Amc (8-aminocaprylyl), Apa (5-aminopentanoyl), Ada (12-aminododecanoyl), AE2A (8-amino-3,6-dioxaoctanoyl), AE4P (15-amino-4,7,10,13-tetraoxapentadecanoyl), ε-Lys(α-NH2) (a Lys residue, the 8-amino group of which is acylated by the carbonyl group of an N-terminally located amino acid; the α-amino group of the Lys residue is free), Agm (agmatine), or absent; and
R³ is Lys(Oct), Ahx (6-aminohexanoyl), or absent.

3. In various aspects, such as the method or use of paragraph 1, the GHRH antagonist is MIA-602, MIA-604, MIA-606, MIA-610, MIA-640, or MIA-690.

4. In various aspects, such as the method or use of paragraph 1, the GHRH antagonist is MIA-602.

5. In various aspects, such as the method or use of any one of paragraphs 1-4, the GHRH antagonist is administered via intradermal, intramuscular, intraperitoneal, intravenous, intraarterial, subcutaneous, epidural, sublingual, intranasal, intracerebral, intraventricular, intrathecal, intravaginal, transdermal, rectally, inhalation, intrapulmonary, intra-airway, intrabronchial, intratracheal, or topical delivery.

6. In various aspects, such as the method or use of paragraph 5, the GHRH antagonist is administered subcutaneously.

7. In various aspects, such as the method or use of any one of paragraphs 1-5, the GHRH antagonist is administered via intranasal, inhalation, intrapulmonary, intra-airway, intrabronchial, or intratracheal delivery.

8. In various aspects, such as the method or use of any one of paragraphs 1-7, the sarcoidosis is pulmonary sarcoidosis.

### BRIEF DESCRIPTION OF THE FIGURES

FIGS. 1A-1E: Bar graphs illustrating the percentage of lung inflammation in mice suffering from sarcoidosis and treated with saline, α-Melanocortin stimulating hormone, (α-MSH, a melanocortin receptor agonist), MIA-602, and Solu-Medrol (methyl prednisolone; current FDA approved medication for sarcoidosis). Pulmonary inflammation was determined and scored by a lung pathologist using Hematoxylin and eosin stain (H&E) staining (FIG. 1A), CD68 levels (FIG. 1B), PD-1 levels (FIG. 1C), PD-L1 levels (FIG. 1D), and CD30 levels (FIG. 1E).

FIGS. 2A-2T: The anti-inflammatory activity of MIA-602 in sarcoid-like granuloma from human cells was tested in peripheral blood mononuclear cells (PBMC) isolated from patients with confirmed sarcoidosis. PBMC were challenged with microparticles resulting in ex vivo granuloma. FIGS. 2A-2T are bar graphs illustrating cytokine production (x-axis, pg/mL) in granuloma samples treated with MIA-602 or Solu-Medrol (methyl prednisolone) with comparison to PBMC not challenged with microparticles and granuloma treated with saline.

FIGS. 3A-3D: Bar graphs illustrating the percentage of levels of Survivin (FIG. 3A), Mcl-1/Bak dimer (FIG. 3B), Bcl-xL/Baak dimer (FIG. 3C) and active caspase-3 (FIG. 3D) in in vitro granuloma samples following treatment with MIA-602, methyl prednisolone, or control.

FIG. 4: Bar graph showing the percentage GHRHR immunofluorescence staining in mice lung with developed sarcoid-like granuloma challenged with microparticles versus granuloma treated with saline or control.

FIGS. 5A-5E: Bar graphs showing H&E (FIG. 5A) and immunohistochemistry (IHC) changes for CD30 (FIG. 5B), CD68 (FIG. 5C), PD-1 (FIG. 5D), PD-L1 (FIG. 5E) in the lungs of mice for control, with granuloma and treated with saline, and granuloma treated with MIA 602. Number of mice: control #3, granuloma #4, MIA 602 #3.

FIGS. 6A-6C: Bar graphs illustrating the percent increase of CD45+CD68+, CD45+CD68+PD-1, or CD45+CD68+PD-L1 cells in granuloma in control mice, mice treated with MIA 602, and mice treated with methyl prednisolone. The percent increase of CD45+CD68+PD-L1 cells was higher after treatment with MIA-602 compared to the other cell types.

FIG. 7: Bar graph showing the percent NOS2 staining in a control mouse lung (without granuloma), the lung of a mouse challenged with microparticles and having developed granuloma, and the lung of a mouse having granuloma and treated with MIA-602.

FIG. 8: Bar graph showing the percent Nitrotyrosine staining in a control mouse lung (without granuloma), the lung of a mouse challenged with microparticles and developed granuloma, and the long of a mouse having granuloma and treated with MIA-602.

FIG. 9: A list of genes which exhibited more than 2.5-fold differential expression upon treatment of sarcoidosis mice with MIA-602. The list includes the gene Stable Identifier (Ensemble database reference number), gene name, and RNA transcript type.

### DETAILED DESCRIPTION

The disclosure provides a method of treating sarcoidosis (e.g., pulmonary sarcoidosis). The method comprises administering a GHRH antagonist to mammalian subject in need thereof. The data set forth herein reveals that GHRH antagonists (e.g., MIA-602) significantly reduces inflammation in an in vivo model of sarcoidosis.

The term “subject” includes, but is not limited to, human and non-human mammals such as wild, domestic and farm animals. Preferably, the subject is a human. The subject may be suffering from any form of sarcoidosis (i.e., sarcoidosis in any organ, such as the lungs).

Growth hormone-releasing hormone (GHRH) is secreted by the hypothalamus and acts on the pituitary gland to stimulate the release of growth hormone (GH). Nearly 2000 synthetic antagonistic analogs of GHRH have been produced by amino acid substitutions in the biologically active N-terminal of GHRH (1-29). Schally et al., Nat. Clin. Pract. Endocrinol. Metab. 4 (1), 33-43 (2008); Zarandi et al., PNAS 91 (25), 12298-302 (1994); Zarandi et al., Peptides 89, 60-70 (2017). The pituitary GHRH receptor (pGHRH-R) is a seven-transmembrane-domain receptor coupled to G-protein. Rekasi et al., PNAS 97 (19), 10561-6 (2000); Havt et al., PNAS 102 (48), 17424-9 (2005). The pGHRH-R, as well as its truncated splice variants (SV) is expressed in various human tissues. SV1 differs from pGHRH-R in the amino-terminal extracellular domain. Rekasi, supra.

In various aspects, the GHRH antagonist is a peptide. Various modifications of GHRH peptides confer antagonistic properties. The GHRH fragment comprising residues 1 to 29, or GHRH(1-29), is the minimum sequence necessary for biological activity on the pituitary. This fragment retains 50% or more of the potency of native GHRH. Many synthetic analogs of GHRH, based on the structure of hGH-RH(1-29)NH₂ peptide have been prepared are contemplated herein for use in the context of the method. hGHRH(1-29)NH₂ has the following amino acid sequence: Tyr-Ala-Asp-Ala-Ile⁵-Phe-Thr-Asn-Ser-Tyr¹⁰-Arg-Lys-Val-Leu-Gly¹⁵-Gln-Leu-Ser-Ala-Arg²⁰-Lys-Leu-Leu-Gln-Asp²⁵-Ile-Met-Ser-Arg²⁹-NH₂ (SEQ ID NO: 1). The GHRH antagonist may comprise a GHRH peptide sequence to which amino acid deletions, insertions, and/or substitutions have been made. The GHRH antagonist may also be a fragment or modified fragment of GHRH having the capability to bind to the GHRH receptor and inhibiting the release of growth hormone. These antagonistic properties are believed to result from replacement of various amino acids and acylation with aromatic or nonpolar acids at the N-terminus of GHRH(1-29)NH₂.

Optionally, the GHRH antagonist is an antagonist described in U.S. Patent Publication No. 20150166617 or U.S. Pat. No. 8,691,942 (incorporated by reference herein in their entirety and particularly with respect to description of GHRH antagonists). For example, in various embodiments, the GHRH antagonist comprises the amino acid sequence (Formula I/SEQ ID NO: 2): R¹-Tyr¹-D-Arg²-Asp³-A⁴-Ile⁵-A⁶-Thr⁷-A⁸-Har⁹-A¹⁰-A¹¹-A¹²-Val¹³-Leu¹⁴-A¹⁵-Gln¹⁶-A¹⁷-Ser¹⁸-Ala¹⁹-A²⁰-A²¹-Leu²²-Leu²³-Gln²⁴-Asp²⁵-Ile²⁶-Nle²⁷-D-Arg²⁸-A²⁹-R²-R³-NH₂, wherein R¹ is PhAc (phenylacetyl), Nac (naphthylacetyl), Oct (octanoyl), N-Me-Aib (N-methyl-alpha-aminoisobutyroyl), Dca (dichloroacetyl), Ac-Ada (acetyl-12-aminododecanoyl), Fer (ferulyl), Ac-Amc (acetyl-8-aminocaprylyl), Me-NH-Sub (methyl-NH-suberyl), PhAc-Ada (phenylacetyl 12-aminododecanoyl), Ac-Ada-D-Phe, Ac-Ada-Phe, Dca-Ada(dichloroacetyl-12-aminododecanoyl), Nac (naphthylacetyl), Nac-Ada, Ada-Ada, or CH₃(CH₂)₁₀—CO-Ada; A⁴ is Ala or Me-Ala; A⁶ is Cpa (para-chlorophenylalanine) or Phe(F)₅ (also known as Fpa5); A⁸ is Ala, Pal (pyridylalanine), Dip ((3,3-diphenyl)alanine), or Me-Ala; A¹⁰ is Fpa5, Tyr(Alk) where Alk is Me or Et; A¹¹ is His or Arg; A¹² is Lys, Lys(0-11) (SEQ ID NO: 9) (i.e., Lys(A0-A1-A2-A3-A4-A5-A6-A7-A8-A9-A10-A11-), where each A is a lysine, otherwise described as a string of lysine residues at position A¹²), Lys(Me)₂, or Orn (ornithine); A¹⁵ is Abu (alpha-aminobutyric acid) or Orn; A¹⁷ is Leu or Glu; A²⁰ is Har (homoarginine) or His; A²¹ is Lys, Lys(Me)₂ or Orn; A²⁹ is Har, Arg or Agm (agmatine); R₂ is β-Ala, Amc (8-aminocaprylyl), Apa (5-aminopentanoyl), Ada (12-aminododecanoyl), AE₂A (8-amino-3,6-dioxaoctanoyl), AE₄P (15-amino-4,7,10,13-tetraoxapentadecanoyl), ε-Lys(α-NH₂) (a Lys residue, the 8-amino group of which is acylated by the carbonyl group of an N-terminally located amino acid; the α-amino group of the Lys residue is free), Agm (agmatine), or absent; and R³ is Lys(Oct), Ahx (6-aminohexanoyl), or absent.

Optionally, the GHRH antagonist is MIA-602: [PhAc-Ada⁰-Tyr¹, D-Arg², Fpa5⁶, Ala⁸, Har⁹, Tyr(Me)¹⁰, His¹¹, Orn¹², Abu¹⁵, His²⁰, Orn²¹, Nle²⁷, D-Arg²⁸, Har²⁹]hGH-RH(1-29)NH₂ (SEQ ID NO: 8), further described in U.S. Patent Publication No. 20150166617 (incorporated herein by reference with respect to the discussion of the structure, activity, and methods of making MIA-602, MIA-604, MIA-606, MIA-610, MIA-640, and MIA-690). Alternative GHRH antagonists include, but are not limited to, Phac-Ada-Tyr¹-D-Arg²-Asp³-Ala⁴-Ile⁵-Phe(F)₅⁶-Thr⁷-Ala⁸-Har⁹-Tyr(Me)¹⁰-His¹¹-Orn¹²-Val¹³- Leu¹⁴-Abu¹⁵-Gln¹⁶-Leu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-Agm-NH₂ (MIA-604/SEQ ID NO: 3); Phac-Ada-Tyr¹-D-Arg²-Asp³-Ala⁴-Ile⁵-Phe(F)₅⁶-Thr⁷-Me-Ala⁸-Har⁹-Tyr(Me)¹⁰-His¹¹-Orn¹²-Val¹³-Leu¹⁴-Abu¹⁵-Gln¹⁶-Leu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-Agm-NH₂ (MIA-606/SEQ ID NO: 4); Phac-Tyr¹-D-Arg²-Asp³-Ala⁴-Ile⁵-Cpa⁶-Thr⁷-Ala⁸-Har⁹-Fpa5¹⁰-His¹¹-Orn¹²-Val¹³-Leu¹⁴-Abu¹⁵- Gln¹⁶-Leu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-Ada-NH₂ (MIA-610/SEQ ID NO: 5); Phac-Ada-Tyr¹-D-Arg²-Asp³-Ala⁴-Ile⁵-Cpa⁶-Thr⁷-Ala⁸-Har⁹-Fpa5¹⁰-His¹¹-Orn¹²-Val¹³-Leu¹⁴-Abu¹⁵-Gln¹⁶-Glu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-Ada-NH₂ (MIA-640/SEQ ID NO: 6); Phac-Ada-Tyr¹-D-Arg²-Asp³-Ala⁴-Ile⁵-Cpa⁶-Thr⁷-Ala⁸-Har⁹-Fpa5¹⁰-His¹¹-Orn¹²-Val¹³-Leu¹⁴-Abu¹⁵-Gln¹⁶-Leu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-NH₂ (MIA-690/SEQ ID NO: 7). The amino acid sequences of the peptides described above are numbered in correspondence with the amino acid residues in hGHRH(1-29) (SEQ ID NO: 1).

The disclosure provides a method of treating sarcoidosis in a subject in need thereof. “Treating” sarcoidosis does not require a 100% remission. Any decrease in sarcoidosis or symptoms of sarcoidosis (e.g., inflammation, granuloma formation, granuloma size), in increase in quality of life, constitutes a beneficial biological effect in a subject. The progress of the method in treating sarcoidosis can be ascertained using any suitable method, such as biomarker detection/measurement in a biological (e.g., blood) sample, chest imaging (e.g., CT-scan), and PET-CT scan. In certain aspects, the method provides a reduction or improvement in a disease indicator, parameter, or symptom, such as a reduction in angiotensin converting enzyme (ACE), SIL2R, or CRP biomarkers, by at least 50%, at least 65%, at least 75%, at least 80%, at least 85%, at least 90%, at least 95% or by at least 99% as compared to pre-treatment, or a reduction in a disease indicator, parameter, or symptom by at least 50% compared to that achieved by treatment with prednisone (administered prior to the instant method or in a matched patient). In various aspects, “treatment” also includes stabilization of the disease, i.e., controlled or no further progression of the disease (e.g., granuloma burden does not increase, or increases by less than 10%, preferably less than 5%, within a given timeframe).

Alternatively, or in addition, treatment as described herein optionally improves the stage of the disease or reduces the severity within a stage. Commonly used stages for sarcoidosis includes: stage I, granulomas located mainly in lymph nodes; stage II, granulomas located in lungs and lymph nodes; stage III, granulomas located mainly in lungs with shrinking lymph nodes; stage IV, pulmonary fibrosis.

Sarcoidosis disease progression is determined using any of a variety of clinical techniques, such as biopsy of the affected organ(s) to identify granulomas, blood test, bronchoscopy, X-ray, neurological tests (e.g., electromyography, evoked potentials, spinal taps, or nerve conduction tests), high-resolution computed tomography (CT) scans, magnetic resonance imaging (MRI), positron electron tomography (PET) scans, pulmonary function tests, and ultrasound.

A particular administration regimen for a particular subject will depend, in part, upon the amount of antagonist administered, the route of administration, and the cause and extent of any side effects. The amount administered to the subject (e.g., human) in accordance with the disclosure should be sufficient to affect the desired response (i.e., ameliorate, prevent or improve an unwanted condition, disease or symptom of a patient) over a reasonable time frame. A therapeutically effective amount of the GHRH antagonist is typically an amount such that when it is administered in a physiologically tolerable excipient composition, it is sufficient to achieve an effective systemic concentration or local concentration in target tissue.

The dose of GHRH antagonist is optionally about 0.005 mg/kg to about 100 mg/kg. In various aspects, the GHRH antagonist is administered in a dose of about 0.05 mg/kg to about 20 mg/kg. In some embodiments, the GHRH antagonist is administered at a dose of about 0.01 mg/kg/dose to about 50 mg/kg/dose, about 0.01 mg/kg/dose to about 25 mg/kg/dose, about 0.1 mg/kg to about 15 mg/kg, or about 1 mg/kg to about 10 mg/kg. Optionally, doses are given once a day or divided into 2-4 administrations/day. When the GHRH antagonist is administered intravenously to human patients, doses are optionally divided into 1-4 bolus injections/day or given as a continuous infusion.

The GHRH antagonist may be administered daily, at least once a week, at least twice a week, at least three times a week, at least four times a week, at least five times a week, six times a week, every two weeks, every three weeks, every four weeks, every five weeks or every six weeks. The treatment period (entailing multiple administrations of the antagonist) will depend on the nature and severity of the disease, as well as the existence of any side effects. Examples of treatment periods include, but are not limited to, 2 weeks, 3 weeks, 1 month, 2 months, 3 months, 4 months, 5 months, 6 months, 9 months, and 12 months.

Methods of administration may include, but are not limited to, oral administration and parenteral administration, including but not limited to, intradermal, intramuscular, intraperitoneal, intravenous, intraarterial, subcutaneous, epidural, sublingual, intranasal, intracerebral, intraventricular, intrathecal, intravaginal, transdermal, rectally, inhalation, intrapulmonary, intra-airway, intrabronchial, intratracheal, or topical (e.g., to the ears, nose, eyes, or skin) delivery. The antagonist is administered subcutaneously in various aspects. In other aspects, such as aspects wherein the subject suffers from pulmonary sarcoidosis, the antagonist is administered via intranasal, inhalation, intrapulmonary, intra-airway, intrabronchial, or intratracheal delivery.

Optionally, the GHRH antagonist is administered either alone or in combination (concurrently or serially) with other pharmaceuticals, optionally as a single, combined formulation or as separate compositions. In some aspects, the method comprises administering multiple GHRH antagonists. Alternatively or in addition, the GHRH antagonist is optionally administered in combination with other anti-inflammatories, such as a steroid. Alternatively or in addition, the GHRH antagonist is optionally administered in combination with one or more disease-modifying antirheumatic drugs (DMARDs; e.g., methotrexate, azathioprine, or leflunomide), a monoclonal antibody (e.g., infliximab, adalimumab, rituximab, or golimumab), colchicine, hormone therapy (e.g., corticotropin), an antibiotic, and/or pentoxifylline.

The GHRH antagonist may be administered in the form of pharmaceutically acceptable, nontoxic salts, such as acid addition salts. Illustrative of such acid addition salts are hydrochloride, hydrobromide, sulphate, phosphate, fumarate, gluconate, tannate, maleate, acetate, trifluoroacetate, citrate, benzoate, succinate, alginate, pamoate, malate, ascorbate, tartarate, and the like. Particularly preferred antagonists are salts of low solubility, e.g., pamoate salts and the like.

Formulations containing the GHRH antagonist and a suitable carrier can be solid dosage forms which include, but are not limited to, softgels, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and powder. In some embodiments, a single dose may comprise one or more administrations (i.e., multiple injections or multiple pills to arrive at a single dose/amount of antagonist).

The GHRH antagonist may be contained in formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted. Pharmaceutical compositions can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

The invention, thus generally described, will be understood more readily by reference to the following example, which is provided by way of illustration and is not intended to limit the invention.

### EXAMPLES

### General Methods

### MIA-602 Preparation

The chemical structure of MIA-602 is [PhAc-Ada⁰, D-Arg², Fpa5⁶, Ala⁸, Har⁹, Tyr(Me)¹⁰, His¹¹, Orn¹², Abu¹⁵, His²⁰, Orn²¹, Nle²⁷, D-Arg²⁸, Har²⁹]hGH-RH(1-29)NH₂. The compound was dissolved in 100% dimethyl sulfoxide (DMSO, ACS grade, Sigma) for stock, and diluted at 1:1000 in corresponding culture medium to a final concentration of 1 μM. Control group in vitro and in vivo received placebo with the same volume and concentration of DMSO.

### Microparticle Development

Microparticles were produced as previously presented (Zhang et al., Sci Rep 2020; 10: 7277). Microparticles were generated from a rough colony of a clinical strain of Mycobacterium abscessus (MAB) with sonicating and heating live bacilli. High quality images of non-infectious, MAB particles were obtained by scanning electron microscope (SEM).

### Human Blood Sample

Blood samples were collected from nine patient with confirmed pulmonary sarcoidosis, randomly selected from the University of Miami Sarcoidosis Biobanking, and matched by age, sex and race with then healthy controls. To avoid the inconvenience and risks associated with additional venipunctures, a 10 ml blood specimen was collected. Patients who currently had a hgb <7 mg/dL were excluded from participating in this study.

### Maturing In-Vitro Granuloma Like Formation

In vitro granuloma was developed by challenging PBMC with microparticles as previously described (Zhang et al., Sci Rep 2020; 10: 7277).

### Mouse Model Exposure to MAB Microparticles

Granulomatous reaction in the mouse lung was developed as previously described (Zhang et al., Front Immunol 2019; 10: 2888).

### ELISA

(Zhang et al., Sci Rep 2020; 10: 7277), PBMCs were lysed in lysis buffer (Cell Signaling Technology, Beverly, MA) with protease inhibitor cocktail (Cell Signaling Technology, Beverly, MA) and sonicated three times for 2 seconds each with at least 1-min rest on ice between each 2-s pulse. Samples were centrifuged at 10,000×g for 5 min at 4° C. and the supernatant was collected. Protein concentration was determined by BCA protein assay kit from Cell Signaling Technology. The methodology is further described in .Zhang et al., Sci Rep 2020; 10: 7277.

Thirty micrograms of total protein were mixed in a reducing sample buffer and used for mitochondrial apoptosis assay per the kit instruction. The assay was performed using a Bio-Rad kit (171-WAR3CK).

To measure cytokines in media, supernatant aliquot samples were analyzed, thawed, and spun at 12,000 rpm for 10 min to separate the particulate material at the bottom. Fifty μl of undiluted media was plated from each sample onto a 96-well V-bottom plate (source plate) by manual pipetting according to predefined maps. The aliquots were wrapped in parafilm and kept in a humid chamber at 4° C. during the entire process, but not longer than 72 hr. Growth factors and their receptor's capture antibodies were reconstituted and diluted per manufacturer specification and 50 μl plated into each well of respective 96-well high-binding half-well plates, which were then sealed and incubated overnight at 4° C. The cytokine levels were measured using a procartaplex human th1/th2 cytokine panel 11 plex from Invitrogen, (epx110-10810-901).

### Immunofluorescence Confocal Microscopy

The detail of the methodology used for confocal microscopy is discussed in Zhang et al., Front Immunol 2019; 10: 2888. To summarize, mice were killed on day 14, and the left lungs were harvested. Lungs were filled with 10% buffered formalin and fixed in formalin for at least 72 h before IHC staining. H&E staining was used to determine inflammatory pathology.

For immunofluorescence, paraffin-embedded serial sections (5 μm) first underwent standard deparaffinization and rehydration procedures, Sections were then probed with GHRHR (Origene, cat #TA311715) as primary antibody, and anti-Rabbit antibody from Sigma, (cat #F-9887) as secondary. Nuclei were counterstained with DAPI. All reagents were from Sigma-Aldrich. Tissue sections were analyzed using fluorescence microscopy and ImageJ software (version 6.0; NIH) to quantitate fluorescent intensity. In trichrome-stained slides, blue stain (collagen content) was also quantitatively analyzed using ImageJ.

Confocal immunofluorescence images were acquired using a Leica DM6000 microscope with a SP5 confocal module at the University of Miami McKnight Analytical Imaging Core Facility. Captured images were processed using Velocity Software version 6.1.1 software (Perkin-Elmer, Waltham, MA).

For immunohistochemistry, 5-μm paraffin sections were processed by deparaffinization and rehydration followed by endogenous peroxidase blocking (1% H₂O₂ in methanol for 20 minutes) and antigen retrieval (boiled in 10 mM citrate buffer for 30 minutes). Tissue sections were blocked with 2% goat or horse serum (Vector Laboratories) and incubated with antibody CD68 (Proteintech, Cat #25747-1-AP), PD-1 (Cell Signaling, cat #84651), PD-L1 (Proteintech, Cat #17952-1-AP), CD30 (Lsbio, cat #LS-c162069) CD3 (Cell Signaling, cat #99940), iNOS (Invitrogen, cat #PAI-036), or Nitrotyrosine (Novus, NBP2-54606) over night at 4° C., washed with TBST five times, then exposed to secondary antibodies (Vector Laboratories, cat #PI-2000). Immunoreactivity was detected using the ABC Elite kit (Vector Laboratories). DAB was used as final chromogen and hematoxylin as the nuclear counterstain. Negative controls for all antibodies were made by replacing the primary antibody with non-immunogen IgG.

Lung inflammation was scored using the three fields with the highest infiltrate's intensity at 100× power magnification as previously described (Zhang et al., Front Immunol 2019; 10: 2888). The area of inflammation was measured and averaged for the three examined high-power fields.

### RNA Isolation and Analysis

RNA from mouse lungs were extracted using RNA Miniprep Plus Kit (Zymo Research). Briefly, whole lung was homogenized in TRI reagent and total RNA extraction was performed following the instructions provided by the manufacturer with additional DNase treatment. Quantity and quality of the samples were determined by NanoDrop spectrophotometer and Agilent Bioanalyzer 2100, respectively (Zhang et al., Front Immunol 2019; 10: 2888).

Preparation and sequencing of RNA libraries was performed. Briefly, total RNA quantity and quality were determined using the Agilent Bioanalyzer. At least 300 ng of total RNA was used as input for the KAPA RNA HyperPrep Kit with RiboErase (HMR) according to manufacturer's protocol to create ribosomal RNA-depleted sequencing libraries. Sequencing was performed on the Illumina NextSeq 500, generating ˜40 million single-end 75 base reads per sample. Sequencing data were processed with a bioinformatics pipeline including quality control, alignment to the hg19 human reference genome, and gene quantification. Count data was inputted into edgeR software for differential expression analysis. Counts were normalized using the trimmed mean of M-values (TMM) method to account for compositional difference between the libraries and paired differential expression analysis using a generalized linear model with sample as a blocking factor. Genes were considered statistically different with a false discovery rate p-value (FDR)≤0.05.

### Flow Cytometry

Mice were sacrificed on day 14, and the left lungs were harvested for pathology after perfusing the right ventricle with 10 ml of PBS.

The upper half of left lung tissue (without trachea, main bronchus and branches) was removed and rinsed by PBS to clean off blood. The tissue was minced and dispersed with a scissors to increase total surface area. To develop single cell suspension, the rubber end of a 5 ml plastic syringe was used to mesh cells through a 100 μm cell strainer with continuous rinse using ice-cold RPMI 1640. Cell suspension was again meshed through a 70 μm cell strainer and rinsed thoroughly with 3 ml of washing buffer containing DNAse followed by 15 ml of DNAse-free washing buffer. The sample was centrifuged at 286×g and 18° C. for 5 min, and the supernatant was discarded (Posel et al. J Vis Exp 2016: 53658).

The cells (10⁶ cells/ml) were resuspended in 100 μl protein blocking solution with 5 μl fluorescent conjugated antibodies, CD8 (Biolegend Cat #100714, CD45 (Biolegend Cat #103130), CD68 (Biolegend Cat #137004), PD-1 (Biolegend Cat #135219), PD-L1 (Biolegend Cat #124308), CD4 (Biolegend Cat #100510), CD11b (Biolegend Cat #101243), CD11c (Biolegend Cat #117318), F4/80 (Biolegend Cat #123146), or IFNg (Biolegend Cat #505836). Samples were analyzed on a BD LSR II flow cytometer using BD FACSDiva software, and data analysis was performed using Flowjo software (TreeStar, Ashland, OR). Cell populations were identified using sequential gating strategy; the expression of activation markers was presented as median fluorescence intensity. Lung immune cells were classified based on FC marker expression as previously described (Misharin et al., Am J Respir Cell Mol Biol 2013; 49: 503-510).

### Example 1

The following example demonstrates treatment of sarcoidosis in vivo using a GHRH antagonist of the disclosure.

A mouse model of pulmonary sarcoidosis was established in C57B1/6 mice by administering microparticles intratracheally. The tongue was pulled out with a small spatula and microparticles were inserted into the trachea using a 20 G angio-catheter tube at the time of laryngeal opening and advanced to main bronchus until reach to resistant. After tube placement, microparticles were administered: a first dose of 5×10⁸ CFU of M. abscessus in 50 μL with three subsequent doses of 2×10⁸ CFU of M. abscessus in 20 μL. A control group received only 20 μL PBS intratracheally. Mice receiving microparticles developed noncaseating granuloma in the lung, as observed use H&E staining and immunohistochemistry staining for CD68 macrophage marker, CD4 marker, and PD-L1.

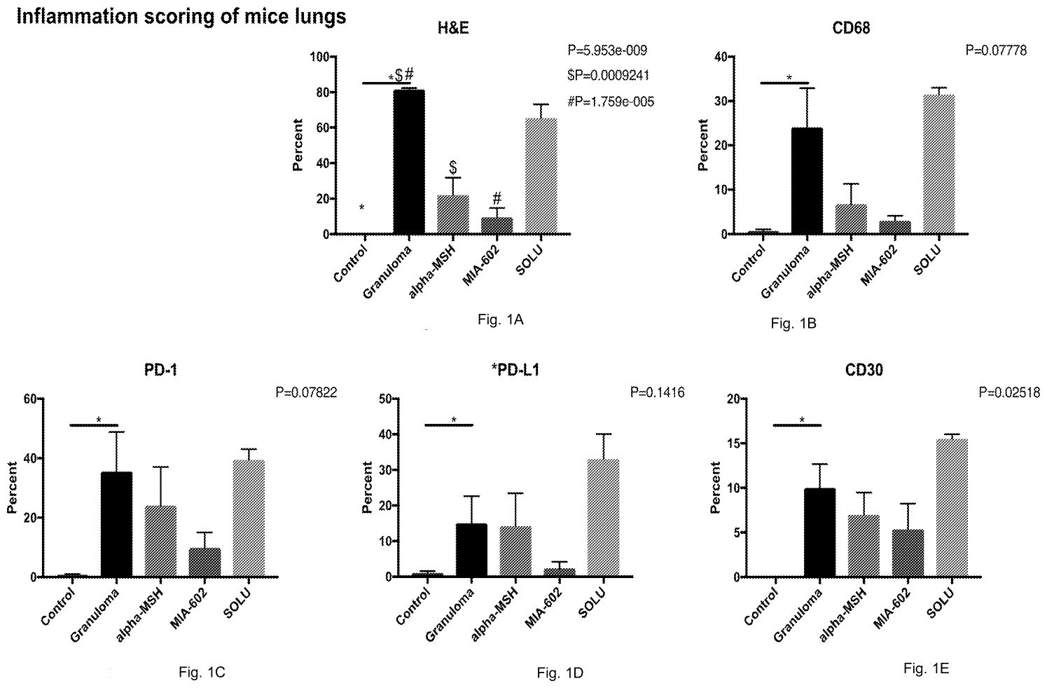

Four groups of mice demonstrating pulmonary sarcoidosis were established alongside a fifth non-treated group which served as a control exhibiting no sarcoidosis. The first sarcoidosis group received MIA-602 (5 μg/day), administered via intraperitoneal injection. The second sarcoidosis group received α-melanocyte stimulating hormone (α-MSH), the third sarcoidosis group received steroid treatment (methyl-prednisone, the current first line treatment for sarcoidosis), and the fourth group received only saline. Mice were sacrificed after two weeks, and inflammation in lung samples was graded. As shown in FIGS. 1A-E, the untreated sarcoidosis group exhibited significant inflammation in the lung. The sarcoidosis groups treated with MIA-602 and a-MSH had lower inflammation scores. Steroid treatment did not affect inflammation.

The results described above demonstrate for the first time that a growth hormone releasing hormone receptor antagonist (here, MIA-602) is effective in treating sarcoidosis.

### Example 2

The following example demonstrates that a peptide of the disclosure positively affects cytokine profiles of treated granuloma.

Granuloma and PBMC were collected from five sarcoidosis subjects. The samples were grouped as control (not challenged), granuloma group challenged with a 10:1 treatment with microparticles but left untreated, challenged granuloma group treated with 1 microM MIA-602 in vitro, and challenged granuloma group treated with methyl prednisolone in vitro. Media were removed 48 h after of treatments. Cytokines were measured using an multiplex ELISA instrument.

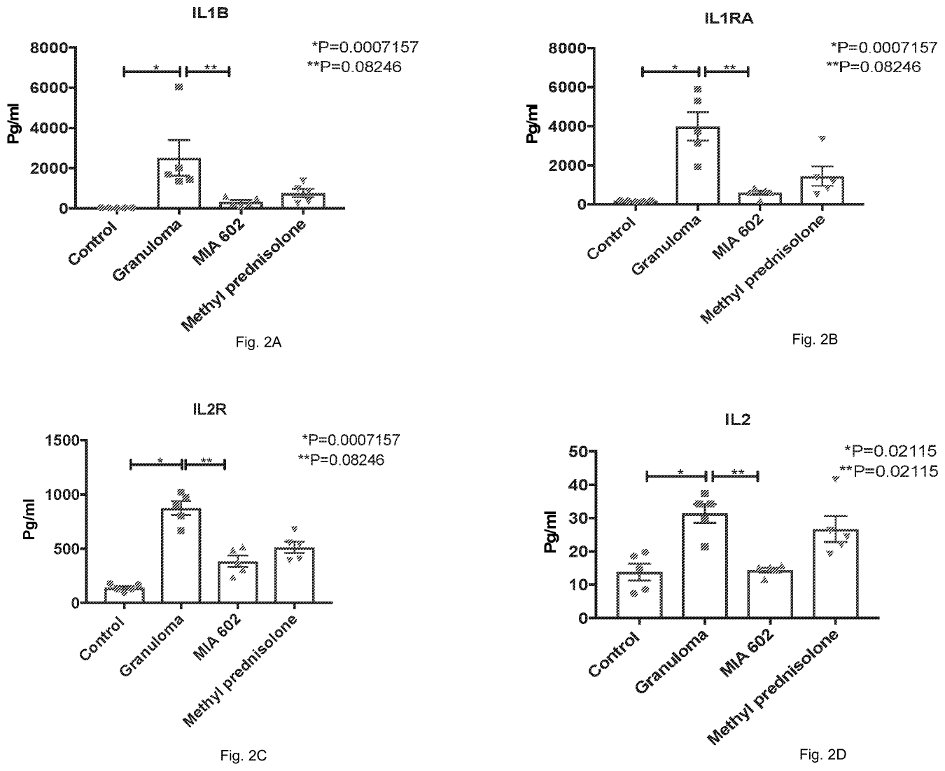

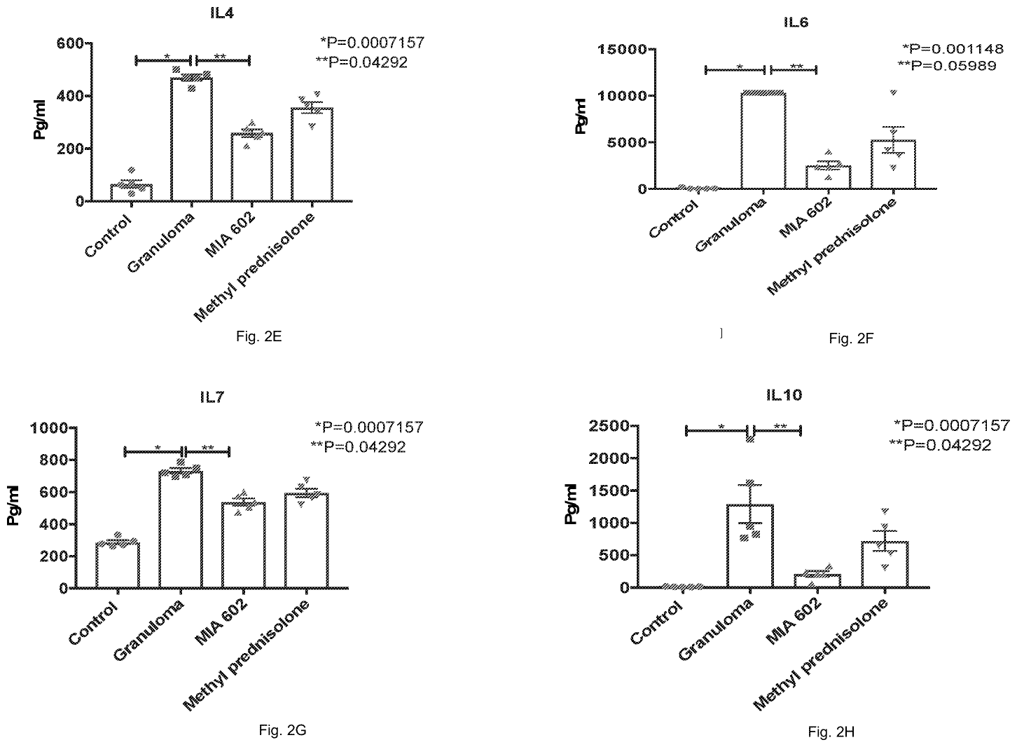

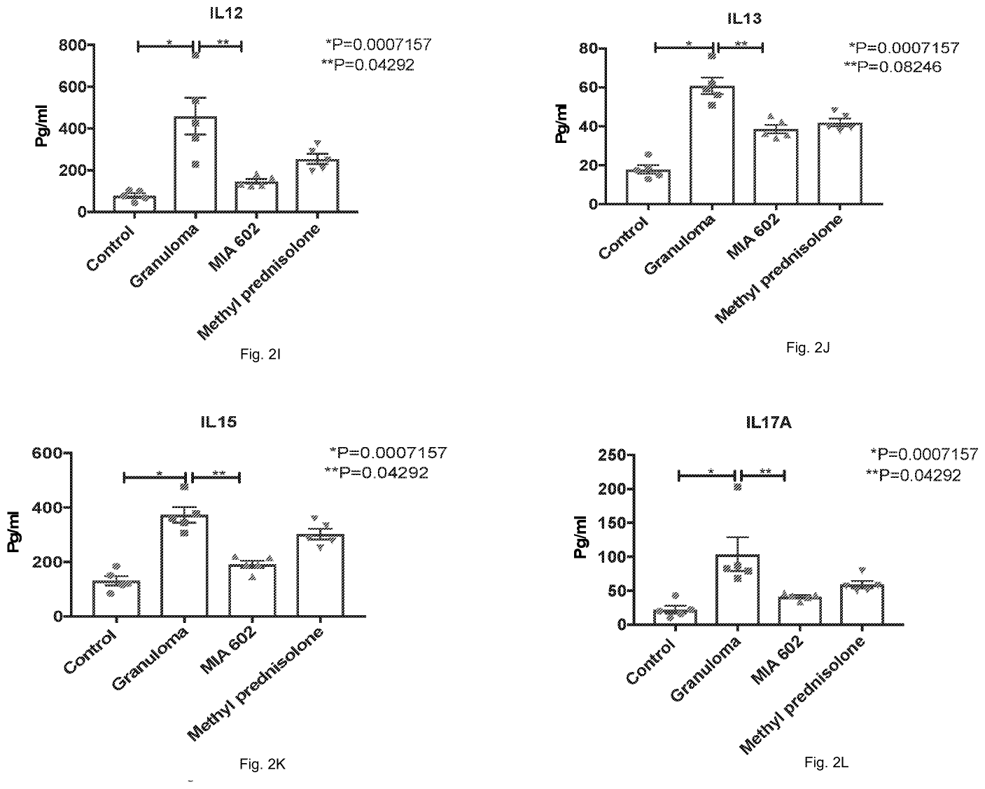

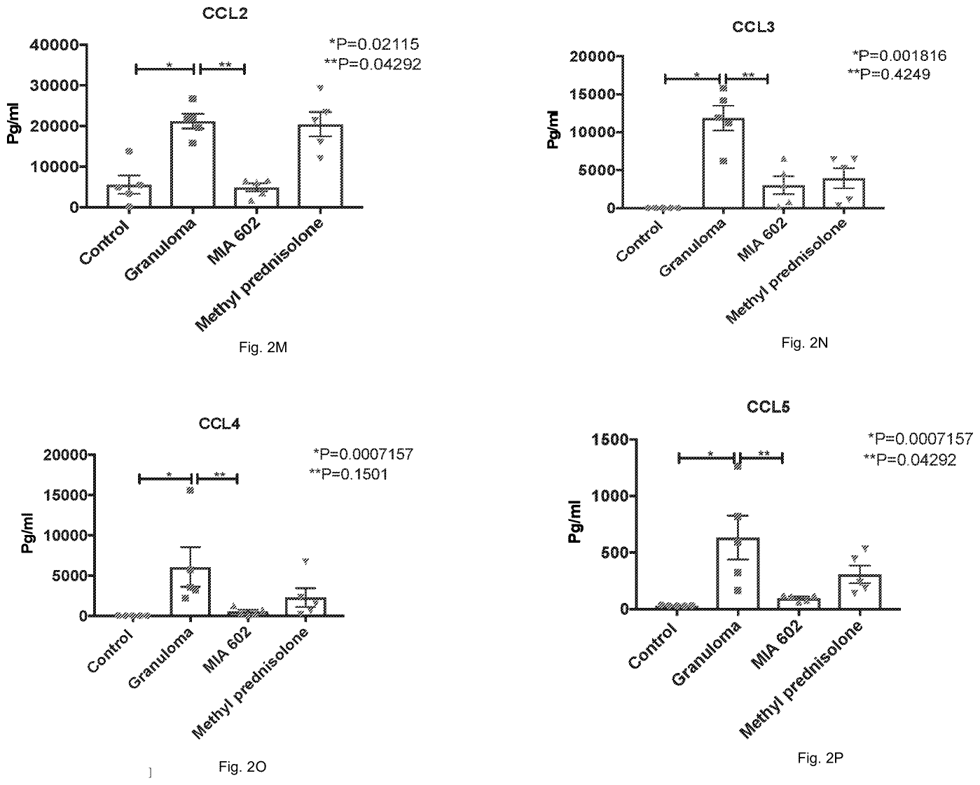

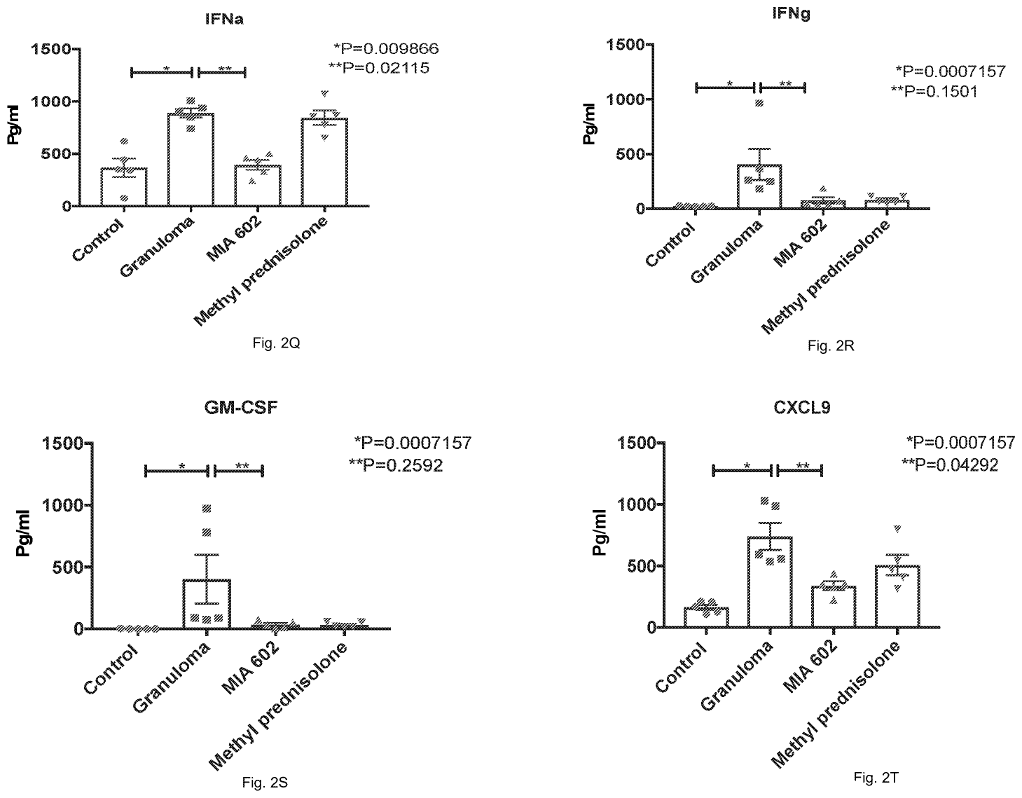

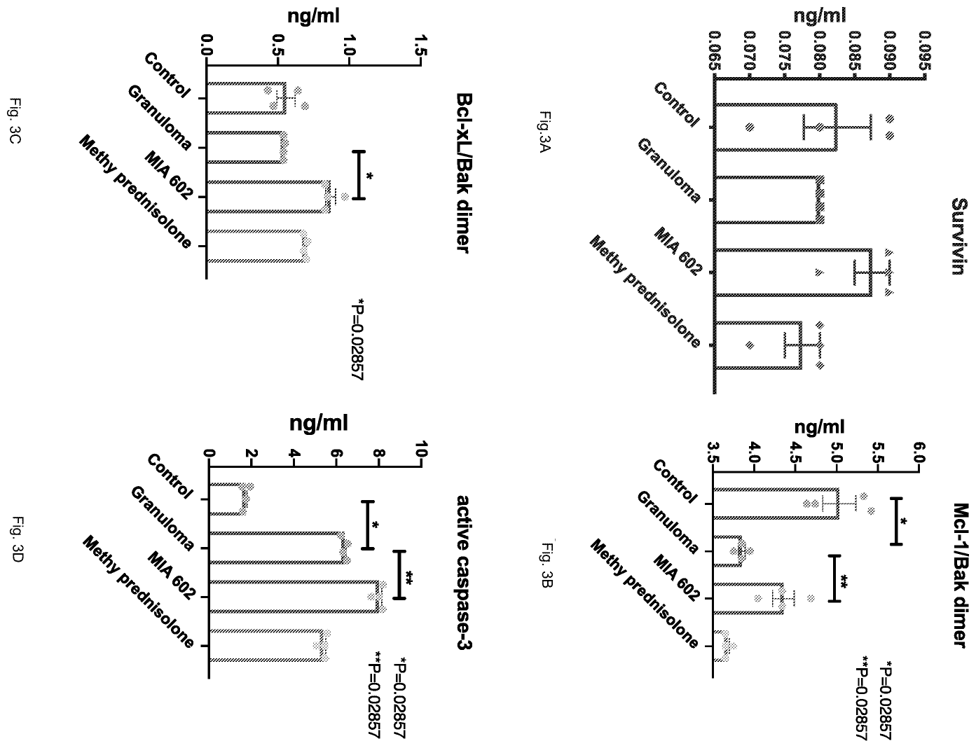

There were significant differences in expression of several cytokines in granuloma in comparison with PBMC. MIA-602 significantly reduced cytokine production of IL2, IL7, IL10, IL12, IL15, IL17A, CCL2, CCL5, IFNα, and CXCL9 in granuloma. See FIGS. 2A-2T.

### Example 3

The ability of a peptide of the disclosure, MIA-602, to influence respiration and apoptosis in granuloma cells was examined. The specific processes of apoptosis and mitochondrial dynamics in granuloma have not been fully elucidated. To test if MIA-602 had pro- or anti-apoptotic effects, the protein levels of pro-(active caspase-3) and anti-apoptotic factors (survivin, Bcl-xL/Bak dimer, and Mcl-1/Bak dimer) were measured in an in vitro granuloma model. PBMCs from five confirmed sarcoidosis subjects were grouped as control (not challenged), granuloma group challenged with 10:1 treatment with microparticles generated from M. abscessus cell wall, granuloma group treated with 1 μM of MIA-602, and a granuloma group treated with methyl prednisolone (138 μM). Media were removed 48 h after treatment.

As shown in FIG. 3A, survivin levels showed no statistical difference between groups. The level of Mcl-1/Bak dimer was significantly reduced in untreated granuloma, but was restored with MIA-602 treatment (FIG. 3B). BclxL/Bak Dimer levels increased in granuloma treated with MIA-602 in comparison with granuloma treated with saline (FIG. 3C). Active caspase 3 level significantly increased in granuloma compared to PBMC, possibly due to lymphocyte early activation (Zhang C et al., Sci Rep 2020; 10: 7277). MIA-602 further increased active caspase 3 (FIG. 3D). These data suggest that treatment with a GHRHR antagonist did not increase apoptosis in granuloma.

### Example 4

A mouse pulmonary granulomatous model was established for studying type I IFN pathways after exposure to M. abscessus cell wall. The model is applicable for pulmonary sarcoidosis studies due to its characteristics of noninfectious lung granulomatous model. C57B1/6 mice were used to develop the model (Zhang et al., Front Immunol 2019; 10: 2888).

Mice were treated with 5 μg of a peptide of the disclosure, MIA-602, per day via intraperitoneal injection. After two weeks, the mice were sacrificed, and lung inflammation was graded by a lung pathologist. Lung samples were stained with H&E, CD68, PD1, PD-L1, and CD30, and scored based on percentage of cells expressing each marker.

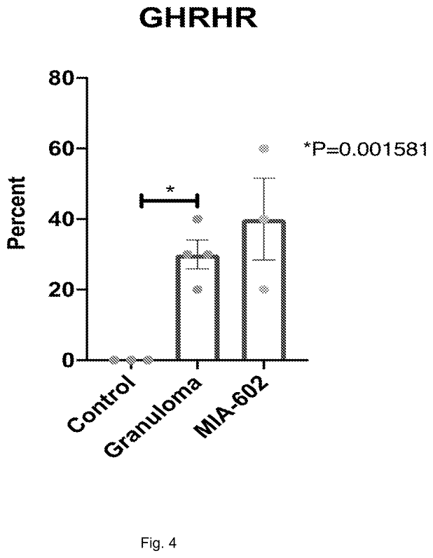

As shown in FIG. 4, the granuloma group had significant inflammation in the lung, but mice treated with MIA-602 had lower inflammation scores. FIG. 4 shows quantification of the GHRHR immunofluorescence staining following MIA-602 treatment in the granulomatous reaction of mice lung.

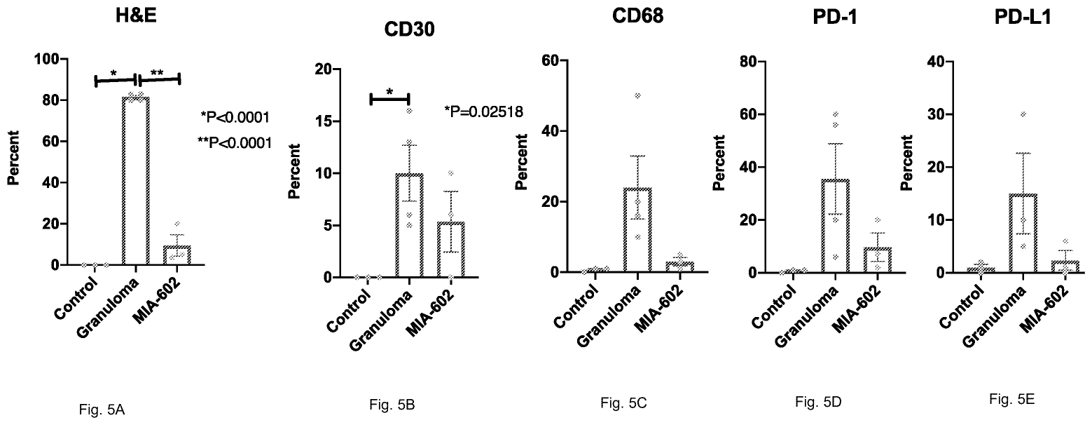

The inflammation score of lungs was significantly higher in the granuloma group. Lung inflammation was almost normalized in mice that received treatment with MIA-602, as shown in FIGS. 5A-5E. CD30 cells were statistically significantly increased in lung with granuloma, and nonsignificantly reduced in MIA-602 treated mice. The percentage of CD68+ cells in the lung increased in granuloma-afflicted subjects and decreased in MIA-602-treated subjects, but the changes were not statistically significant. This pattern was seen in PD-1+- and PD-L1+ cells in the granuloma group and the MIA-602-treated group.

This animal model study confirmed that GHRHR significantly increased in sarcoidosis lung. MIA-602 has anti-inflammatory properties that reduced inflammatory cell numbers significantly in lung tissue.

### Example 5

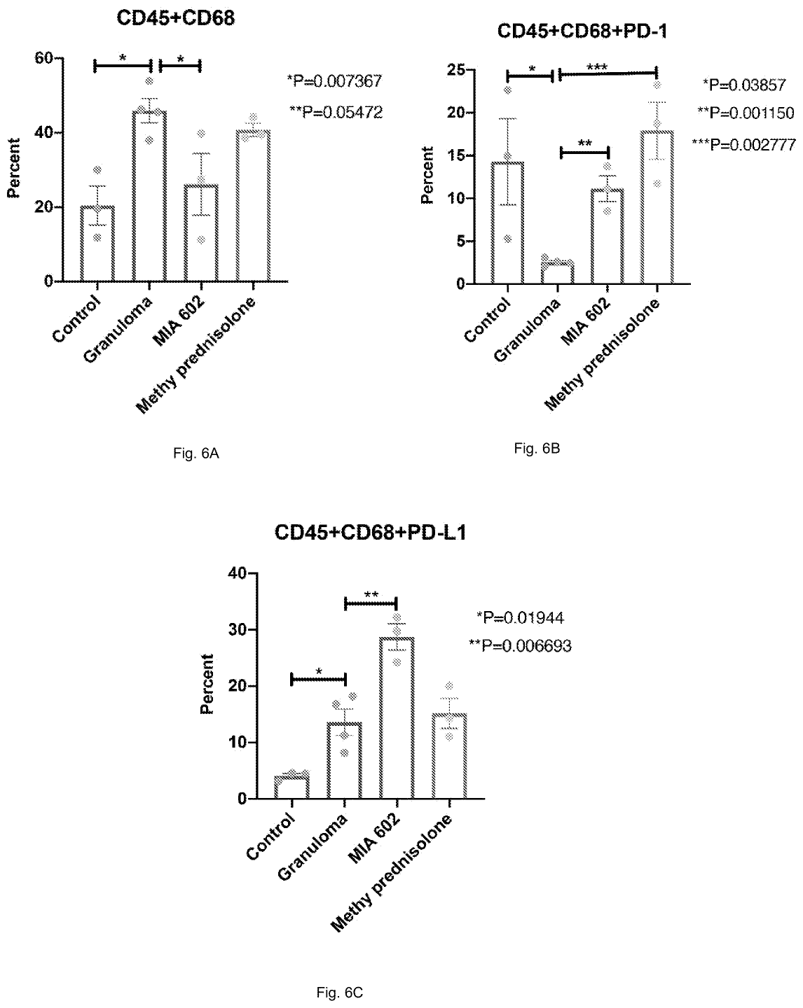

Flow cytometry showed that CD68⁺ cells reduced after MIA-602 therapy in the sarcoidosis mice model. Mice were grouped as control, challenged with microparticles and treated with saline, challenged and treated with MIA-602 (5 μg), and challenged and treated with methyl prednisolone (100 μg). After two weeks, lungs were harvested from all groups, and lung single cells were generated for flow cytometry analysis. As shown in FIG. 6A, the population of CD45+CD68 cells significantly increased in challenged mice, and the population was significantly reduced by MIA-602 treatment.

It was hypothesized that MIA-602 will restore the number of CD68+ cells that express PD-1. FIG. 6B shows that the number of CD45+CD68+ cells that expressed PD-1 were significantly reduced in challenged mice with granulomatous reaction in lungs. The percentage of these cells significantly increased following MIA-602 treatment.

To confirm that mice lung with granuloma shows higher percentage of CD45+CD68+ that expressed PD-L1, lung single cells were stained in all experimental groups. As shown in FIG. 6C, the population of CD45+CD68+PD-L1 cells significantly increased in granuloma, and a higher percentage was detected after treatment with MIA-602. The anti-inflammatory effects of MIA-602 were mainly through reduction in CD68+ cells, and PD-1 and PD-L1 play role on this process.

### Example 6

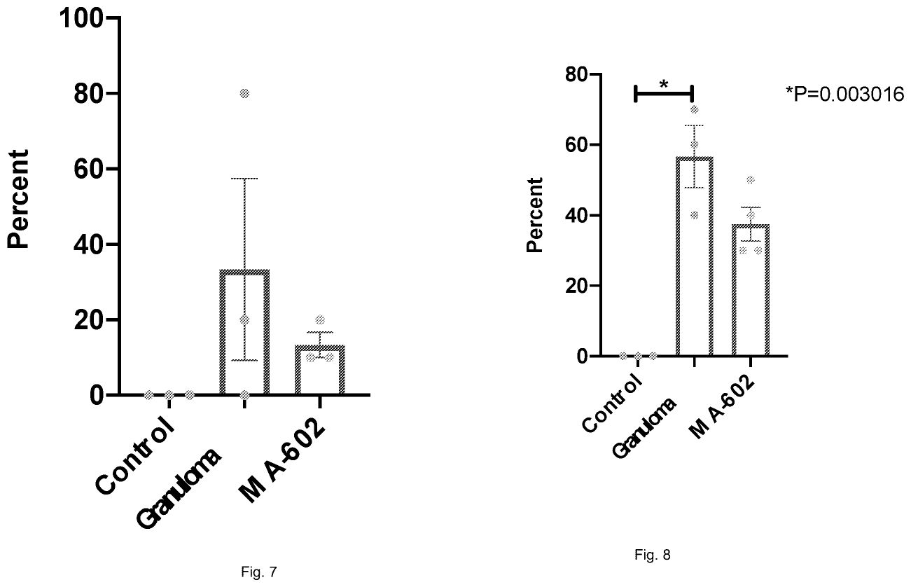

Inducible nitric oxide synthases (iNOS) produces nitric oxide and has a crucial role in granuloma development. iNOS is expressed in macrophages after exposure to bacterial lipopolysaccharides and IFNγ (Facchetti et al., Am J Pathol 1999; 154: 145-152). To understand MIA-602's effects on nitric oxide response, iNOS and nitrotyrosine (as an indicator of NO function) were detected by staining mice lung challenged with microparticles (sarcoidosis model) and injected with saline daily, those that were treated with daily intraperitoneal injection of MIA-602 (5 μg), and a control group which were not challenged and did not receive MIA-602. Lung tissue was harvested after three weeks and tissue stained with NOS2 and nitrotyrosine. As shown in FIG. 7, NOS2 expression was increased in sarcoid-like granuloma and then reduced following MIA-602 treatment. The finding was not statistically significant.

Nitrotyrosine expression was statistically significantly increased in the lung of mice with sarcoid-like granuloma as shown in FIG. 8. Treatment with MIA-602 reduced the nitrotyrosine levels, but the reduction was not statistically significant.

Challenge with microparticles activated iNOS and increased nitrotyrosine in the lungs. MIA-602 reduced both, but not in a statistically significant manner, in these experiments. These experiments suggest an anti-nitrosative effects of MIA-602 in sarcoidosis mice model.

### Example 7

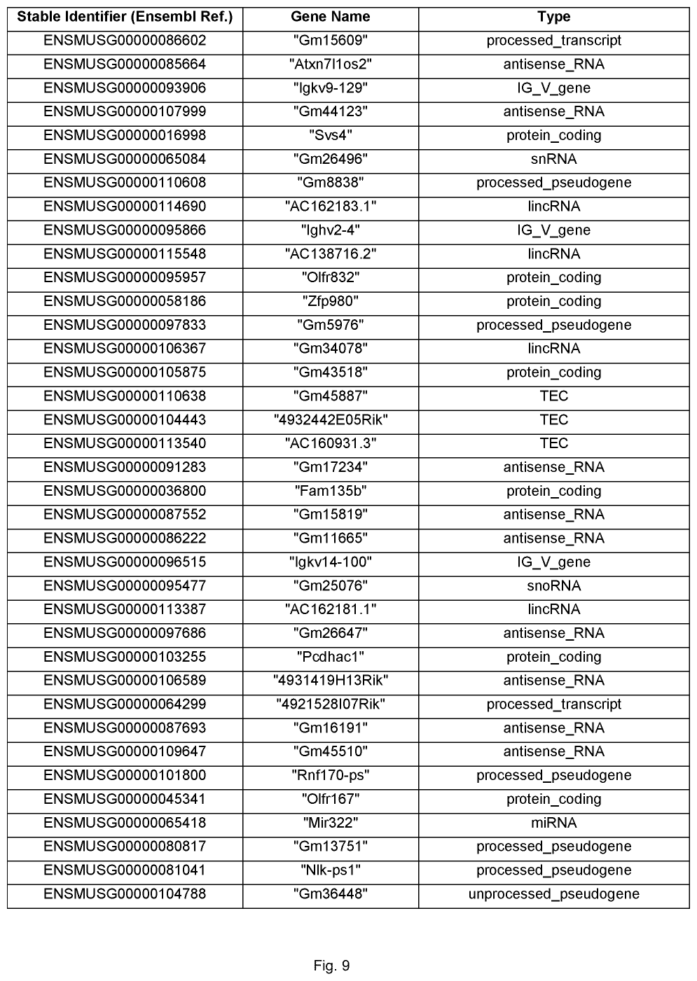

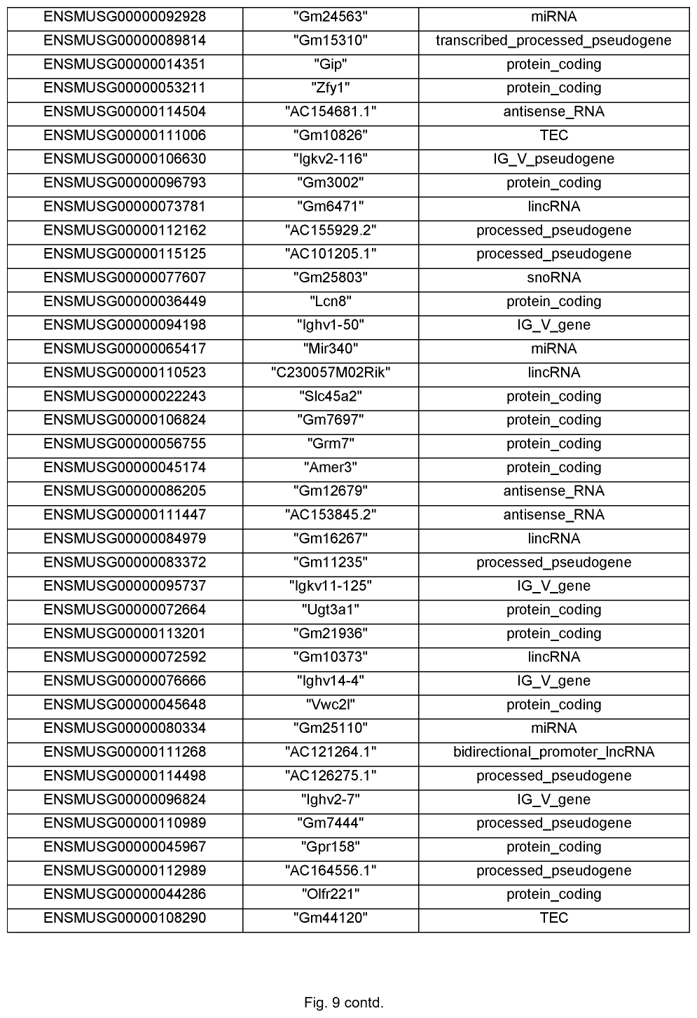

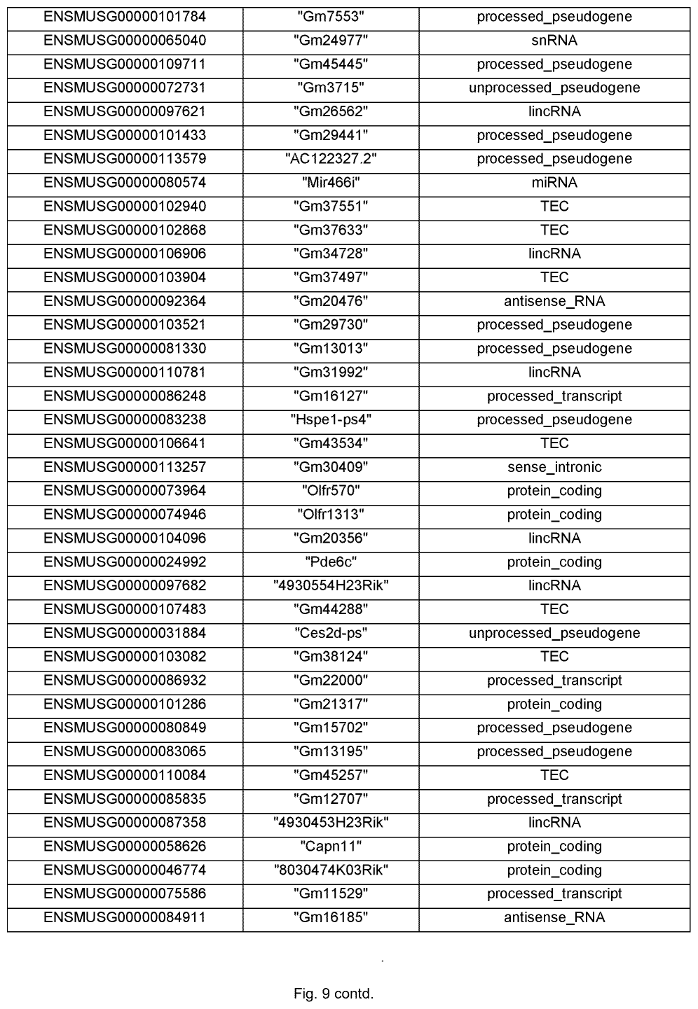

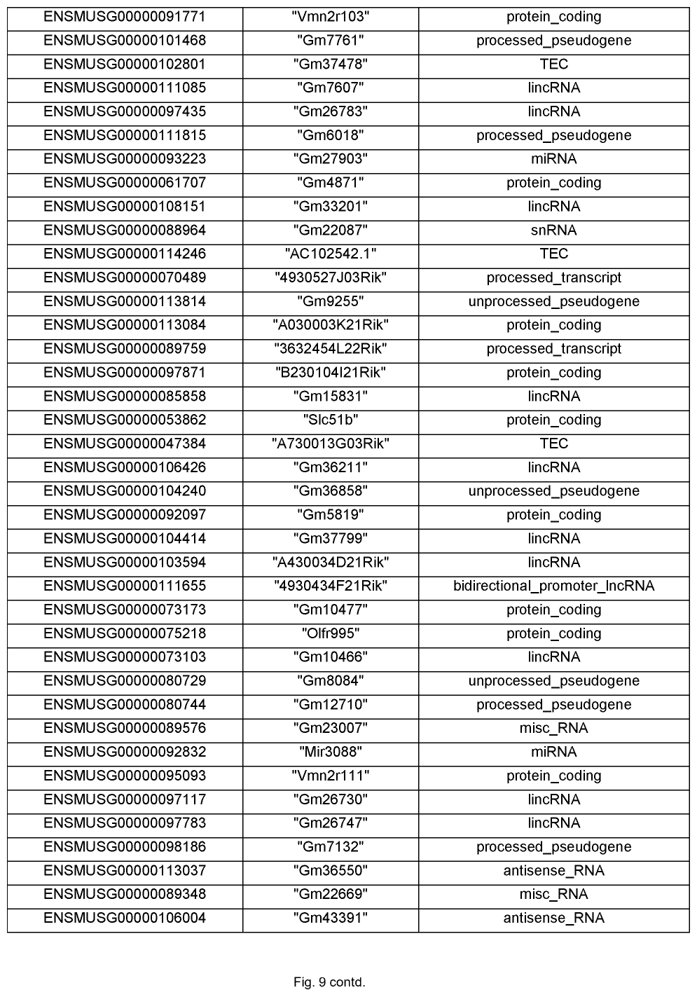

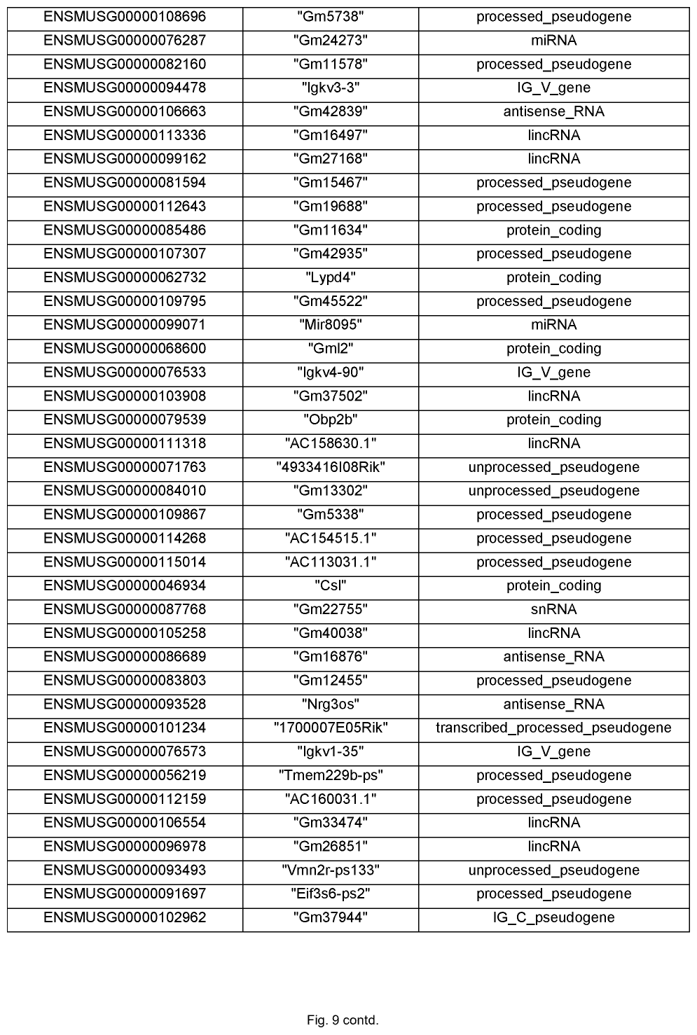

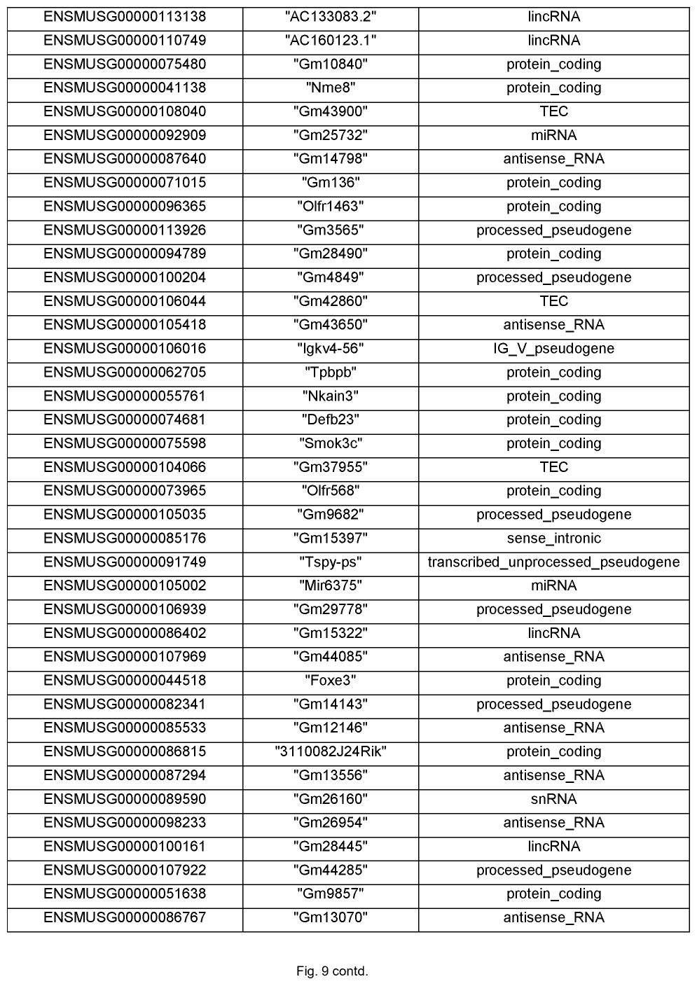

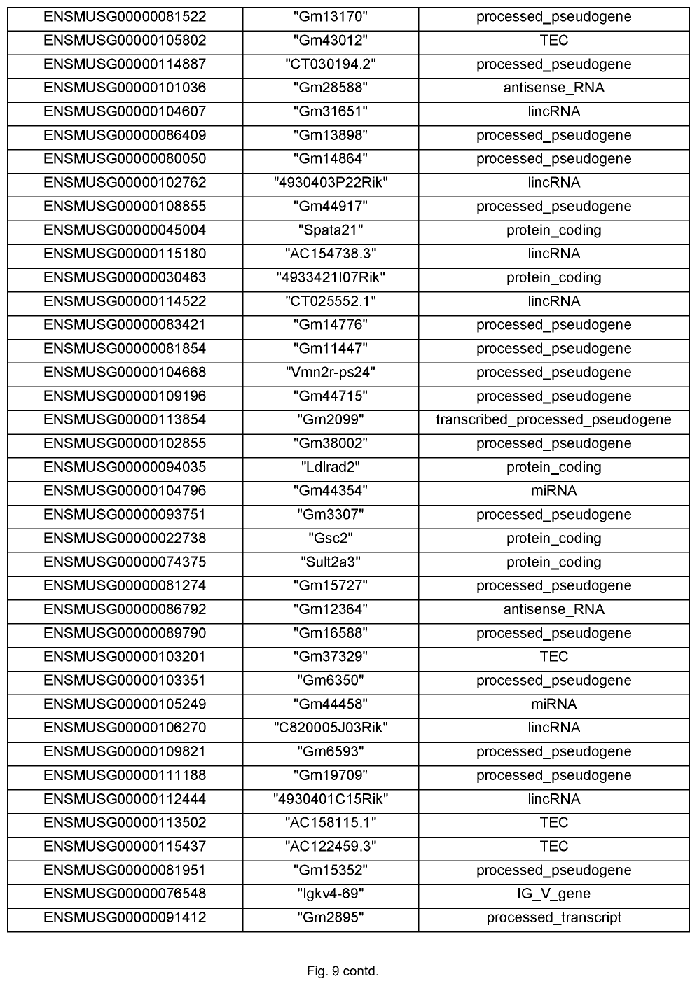

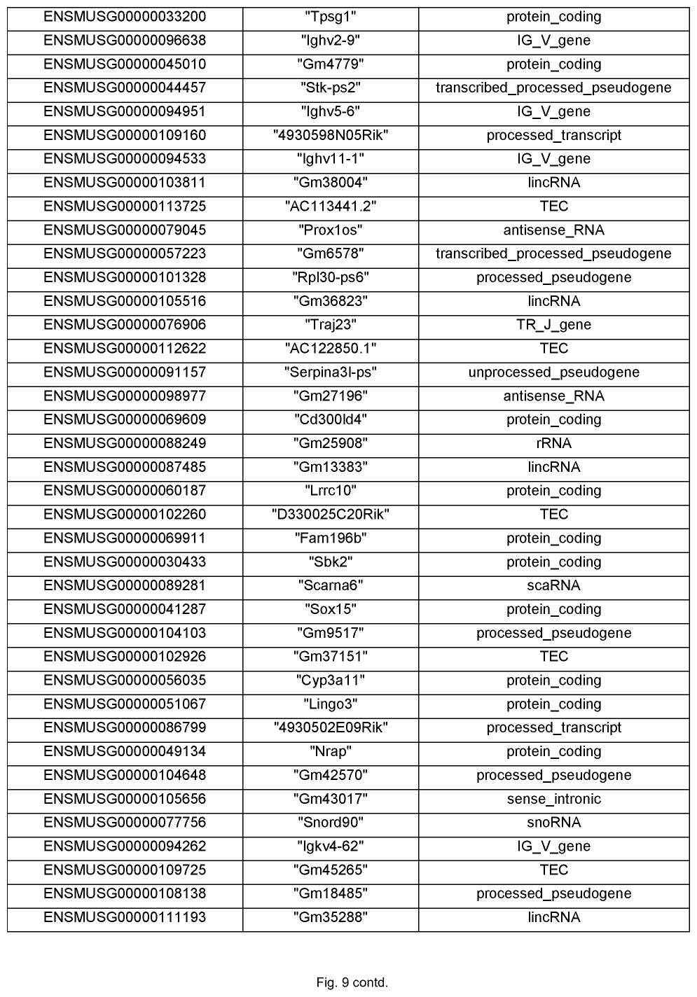

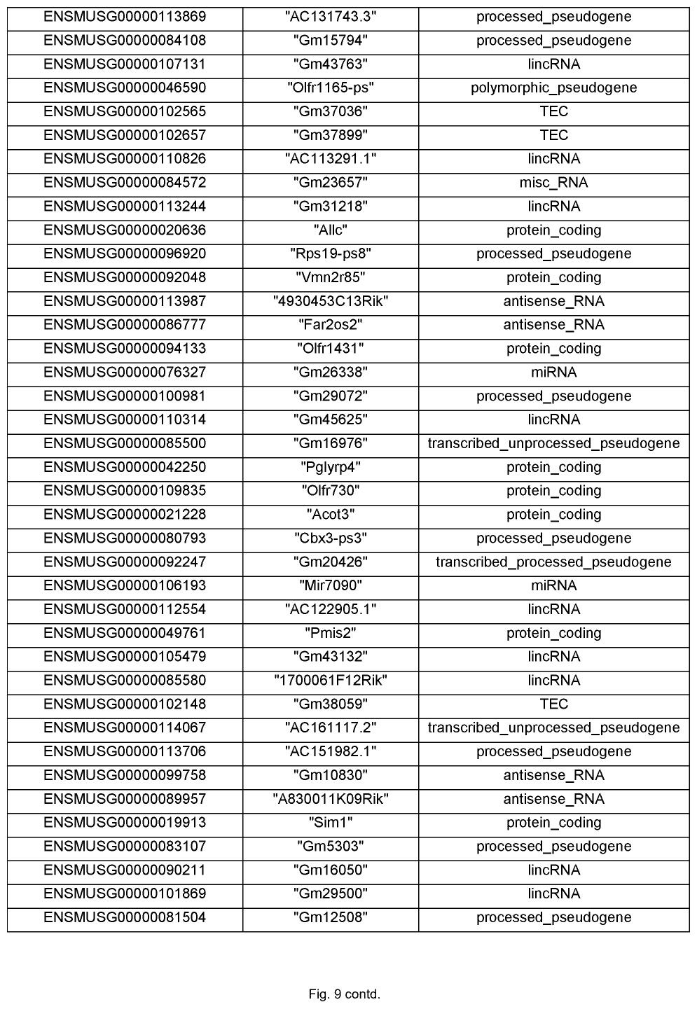

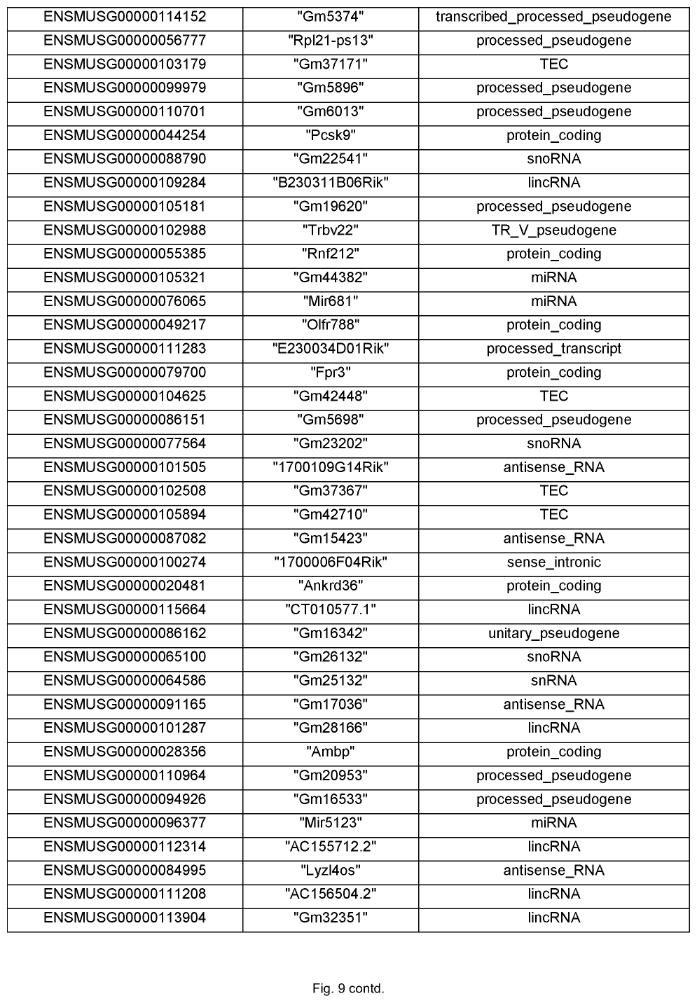

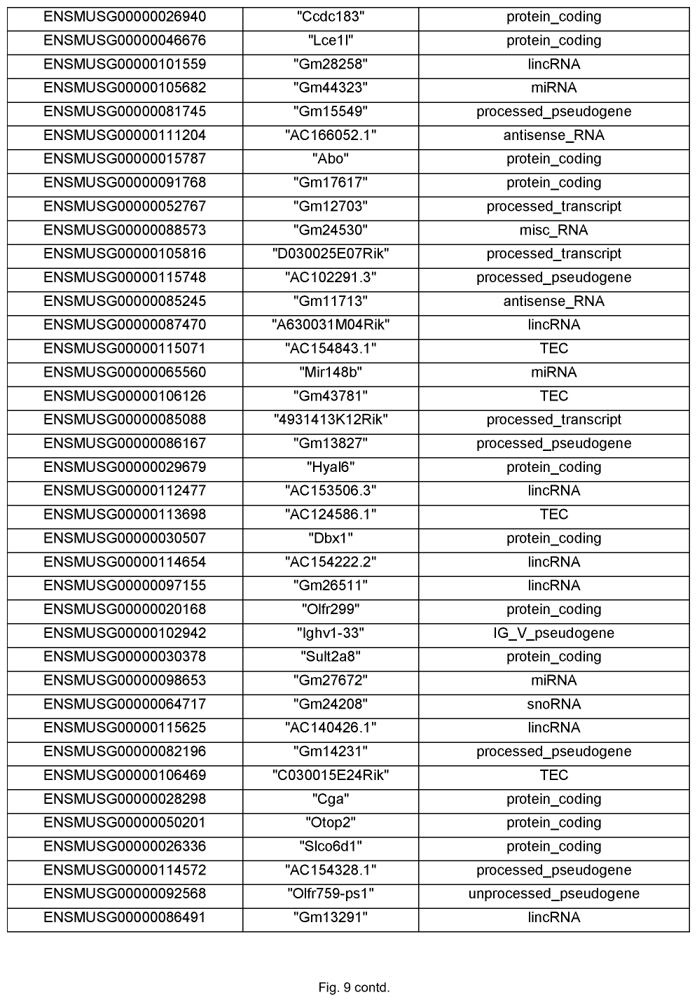

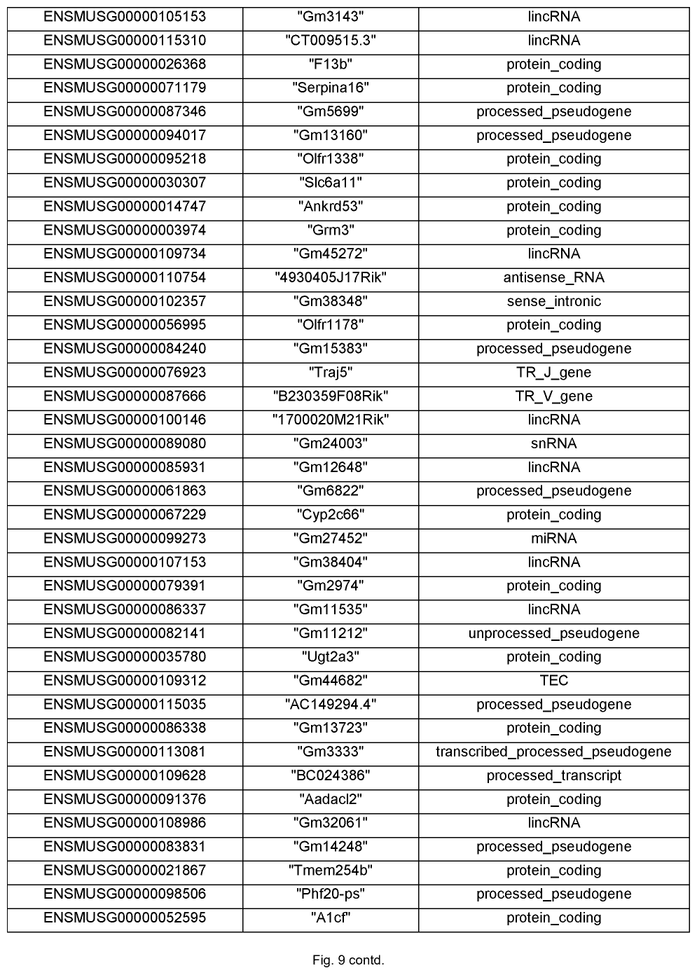

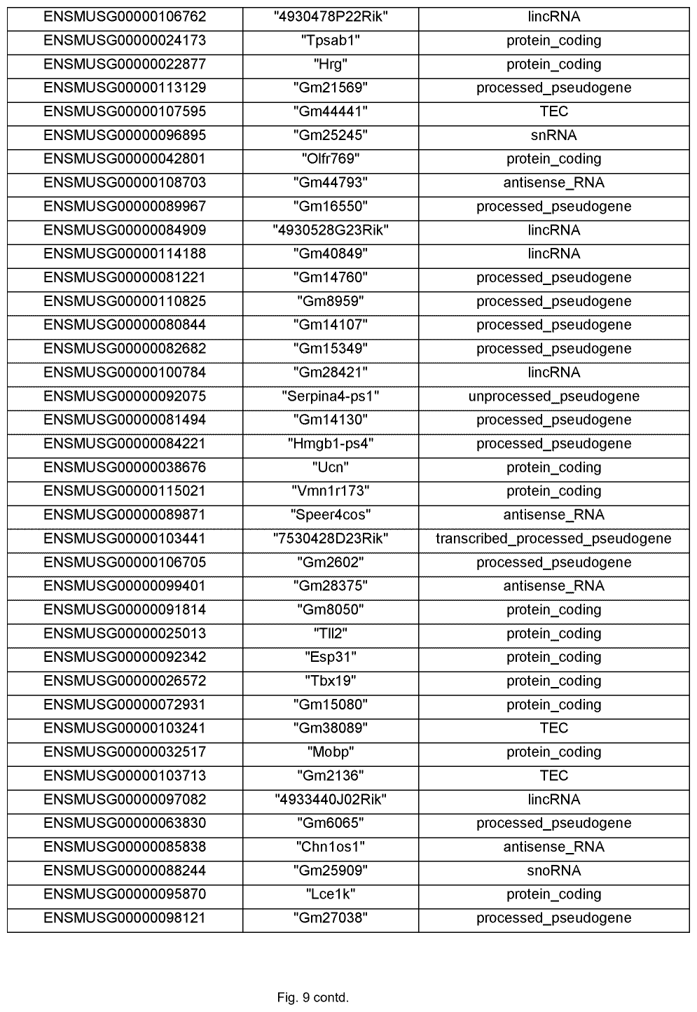

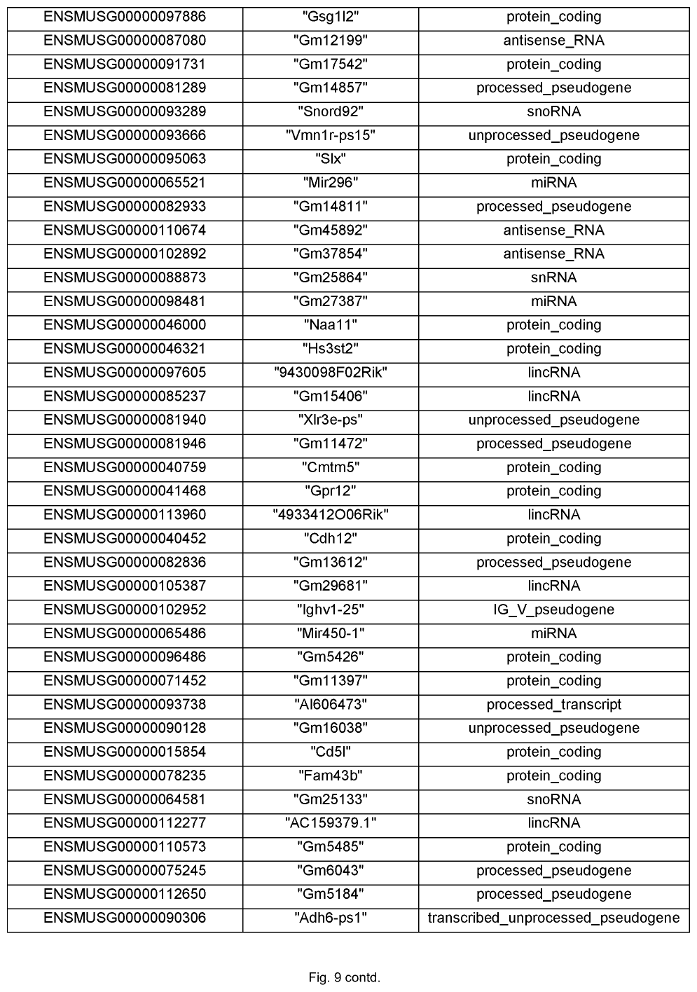

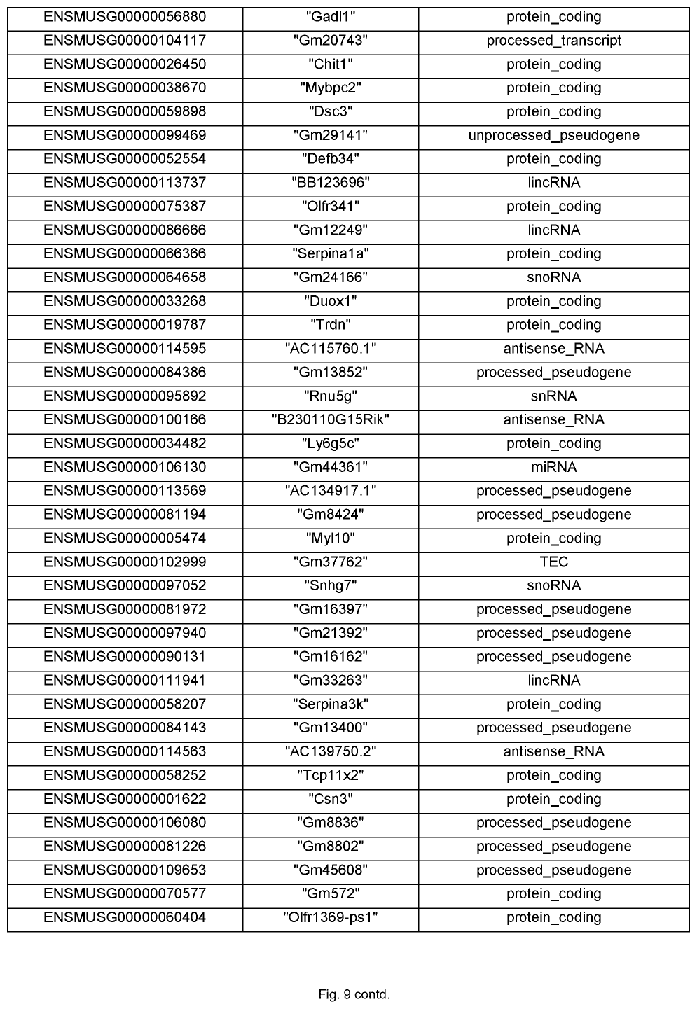

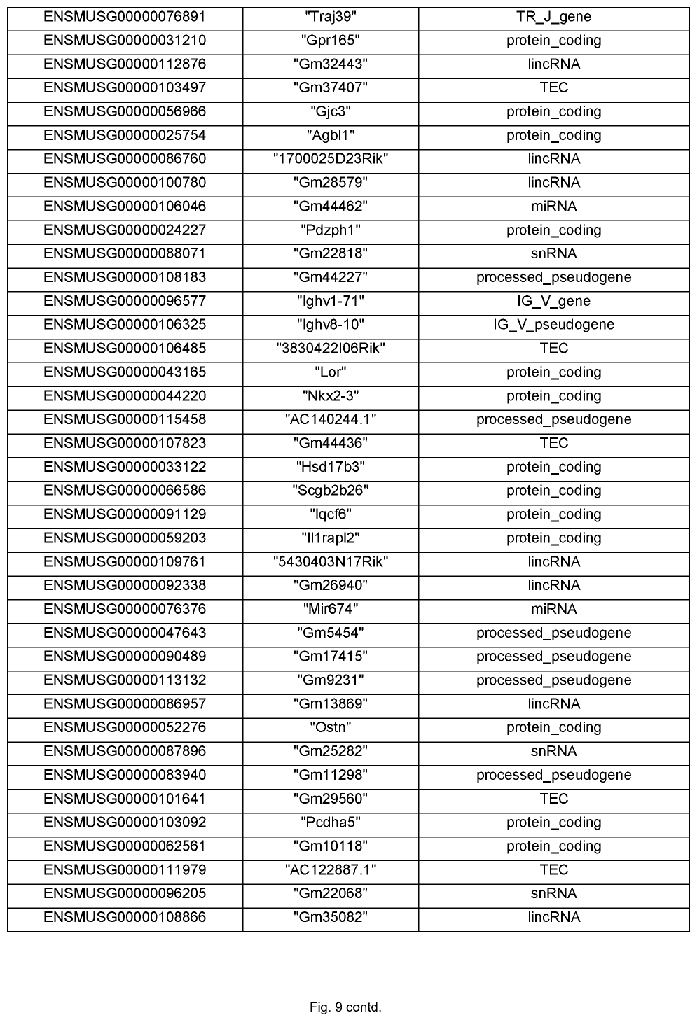

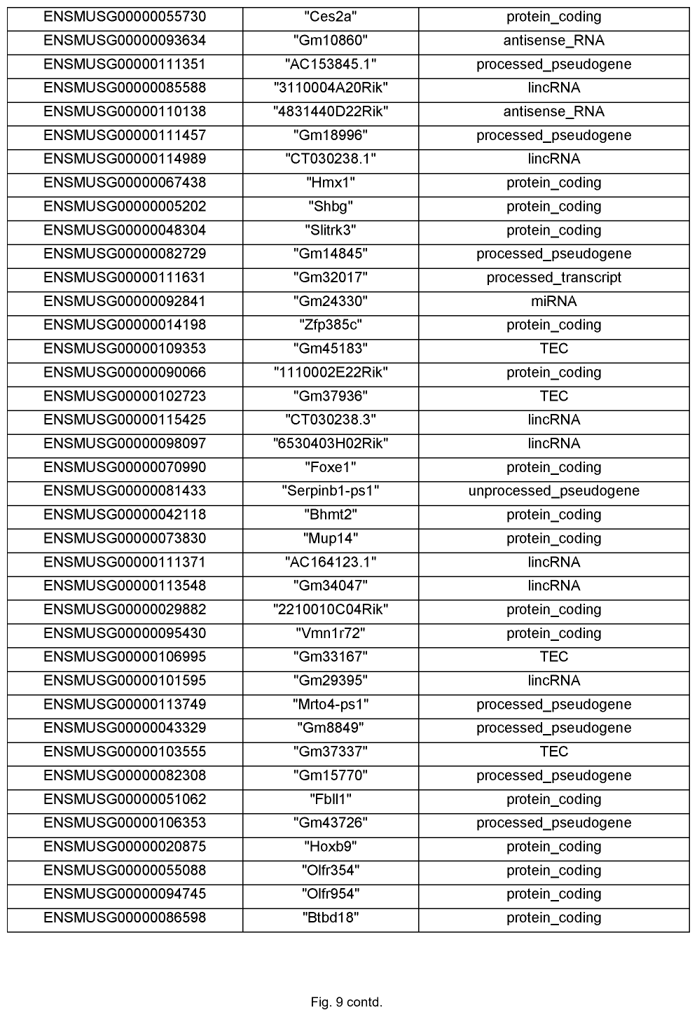

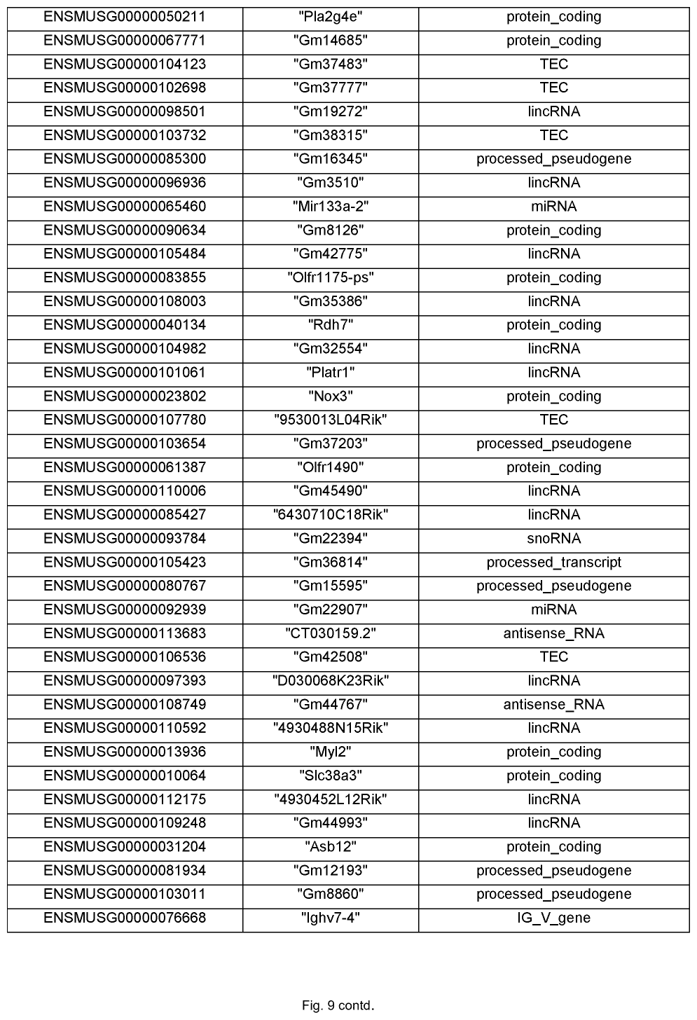

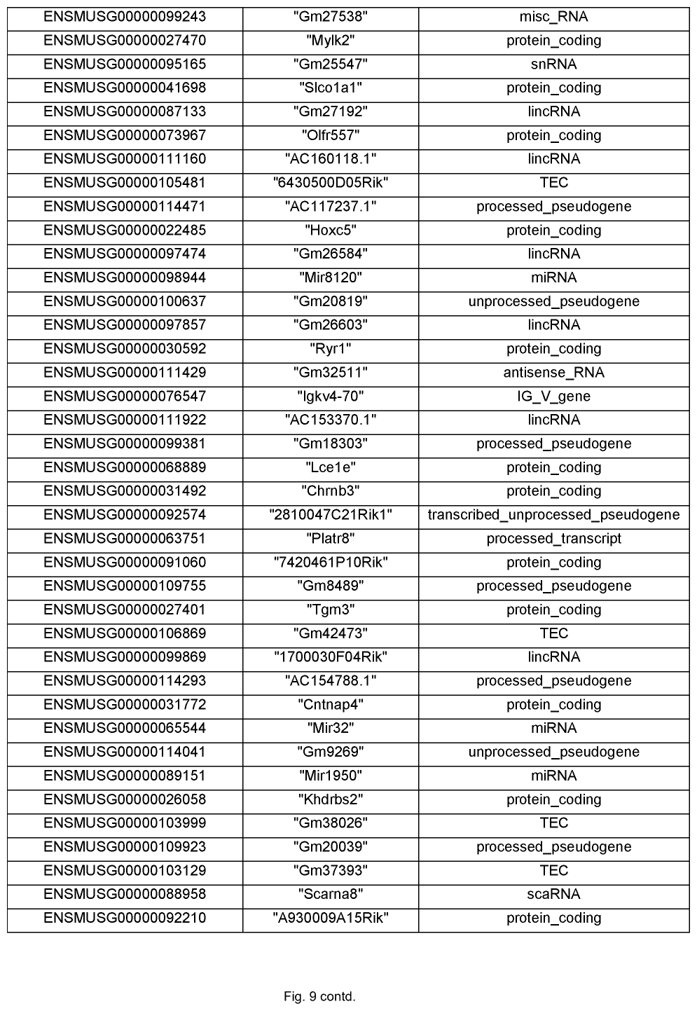

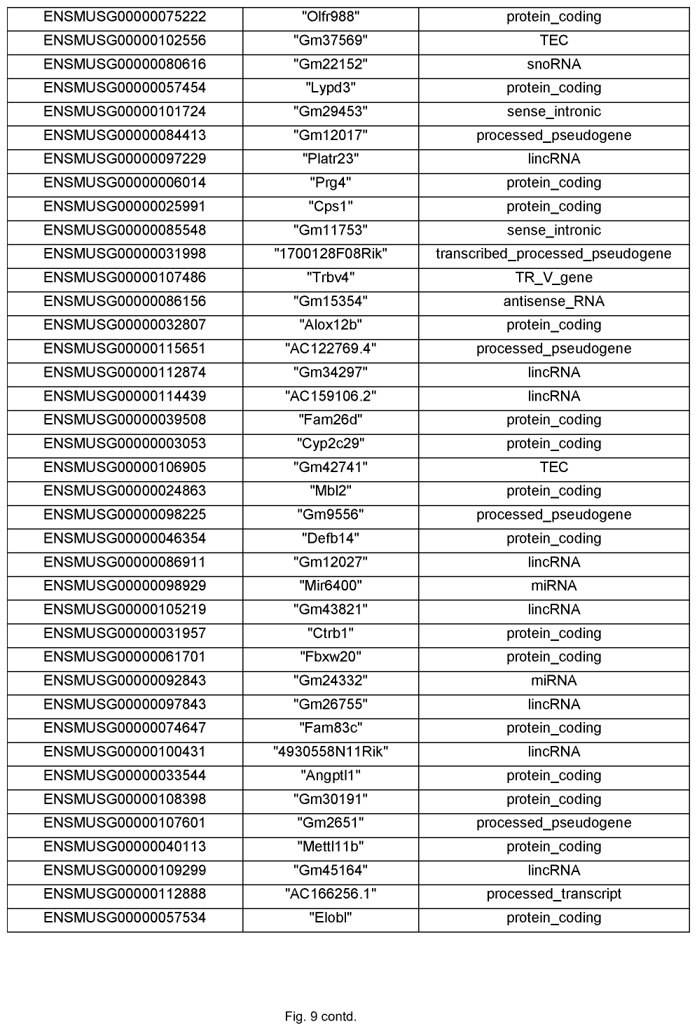

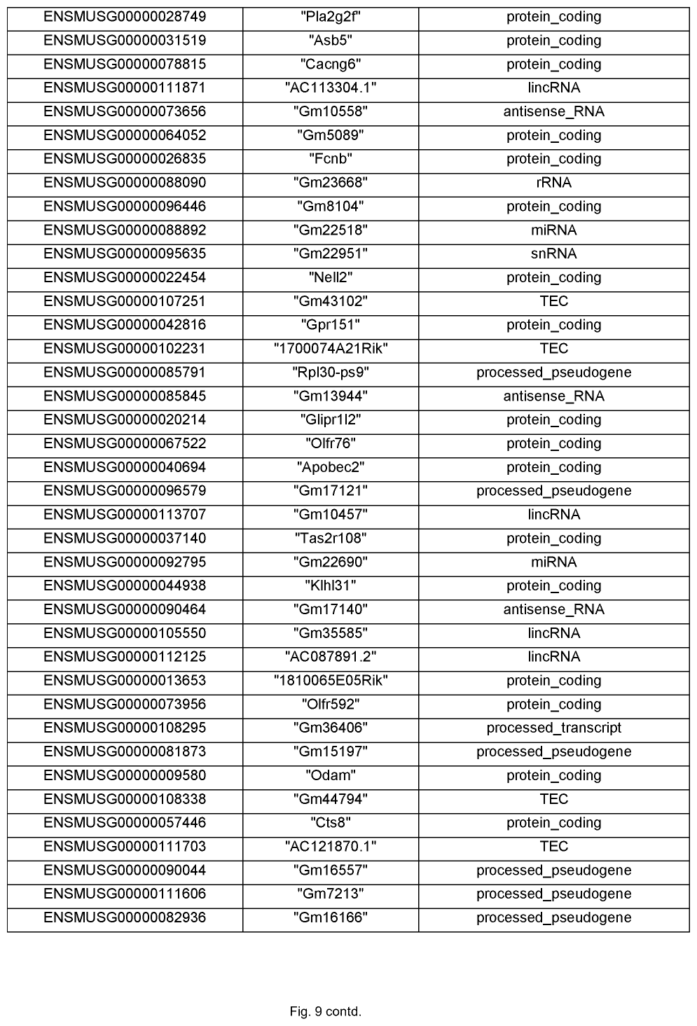

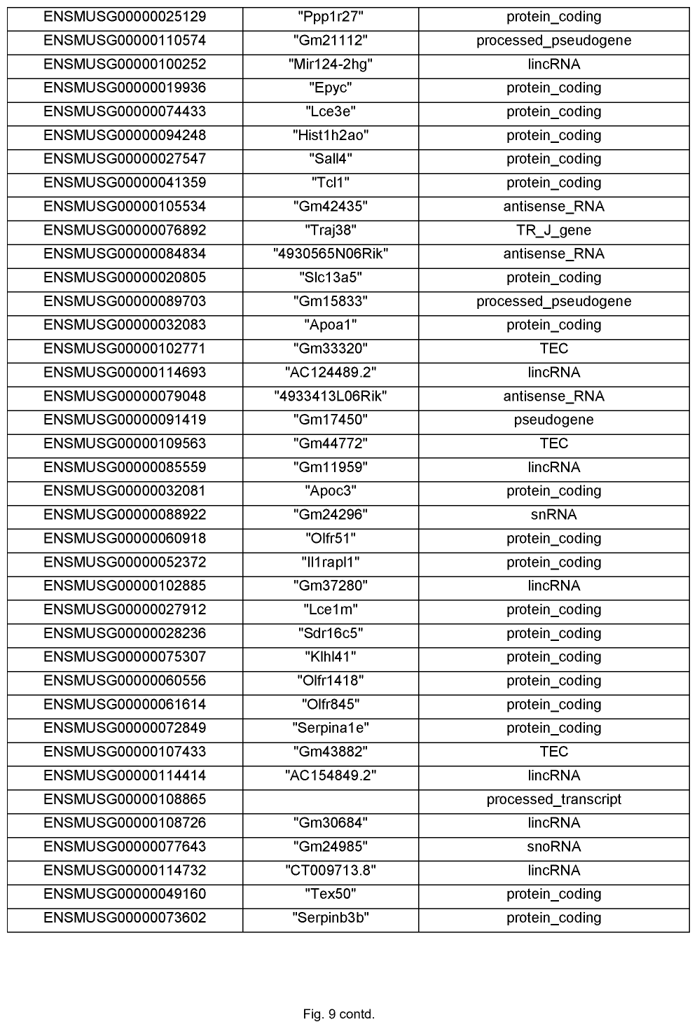

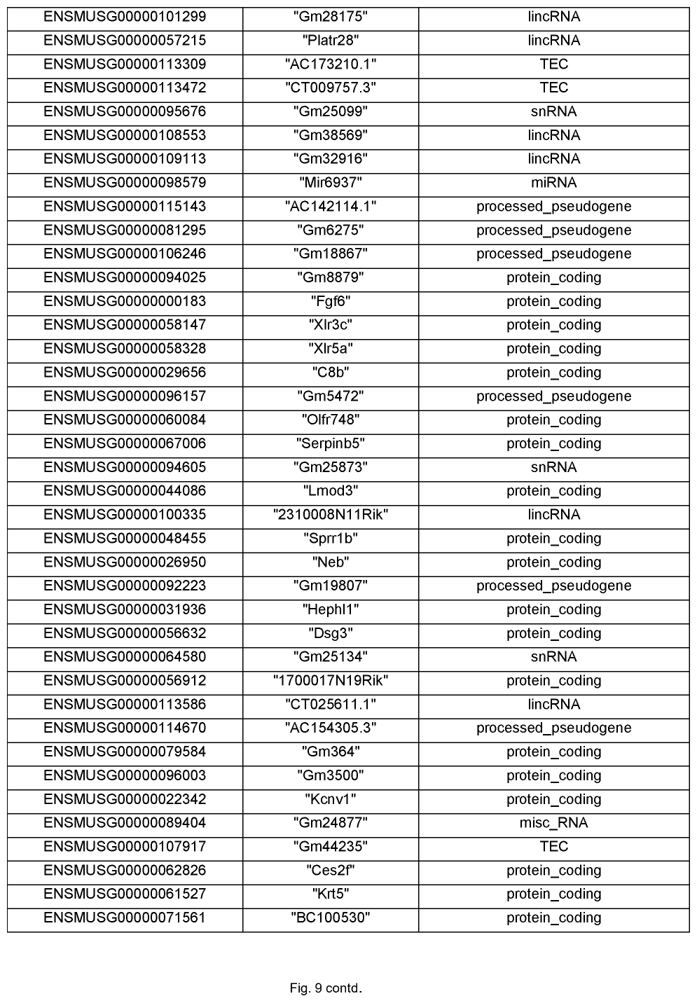

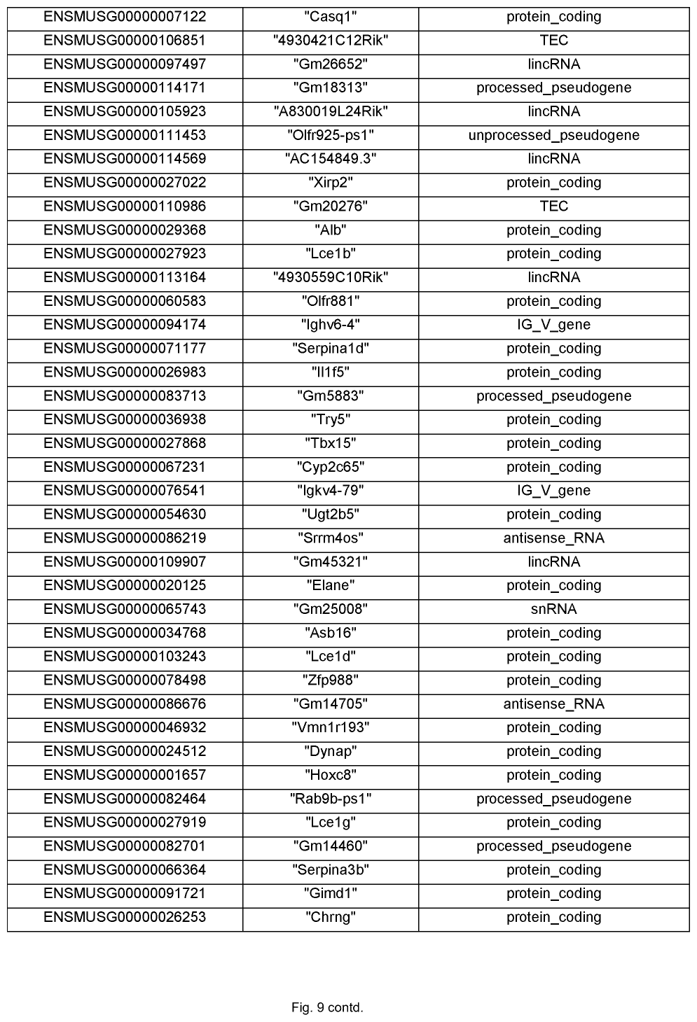

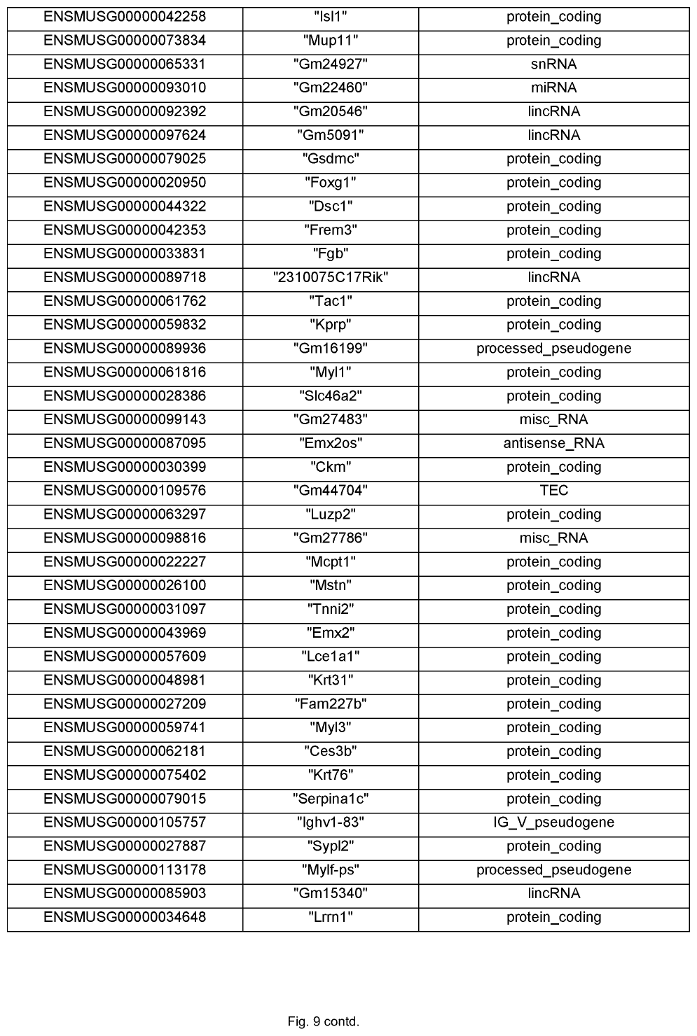

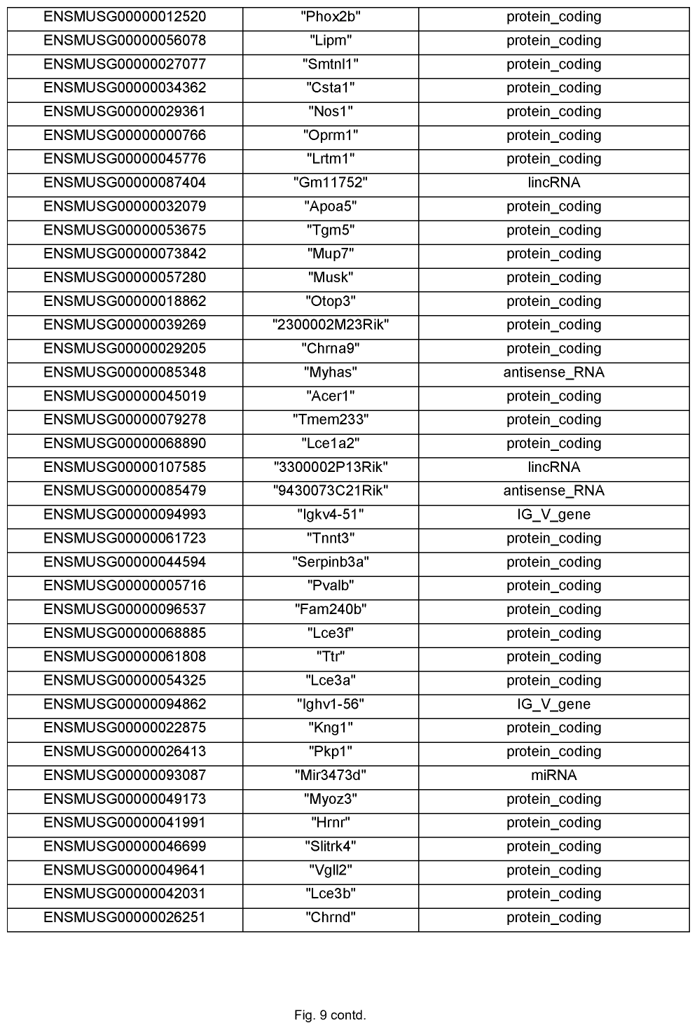

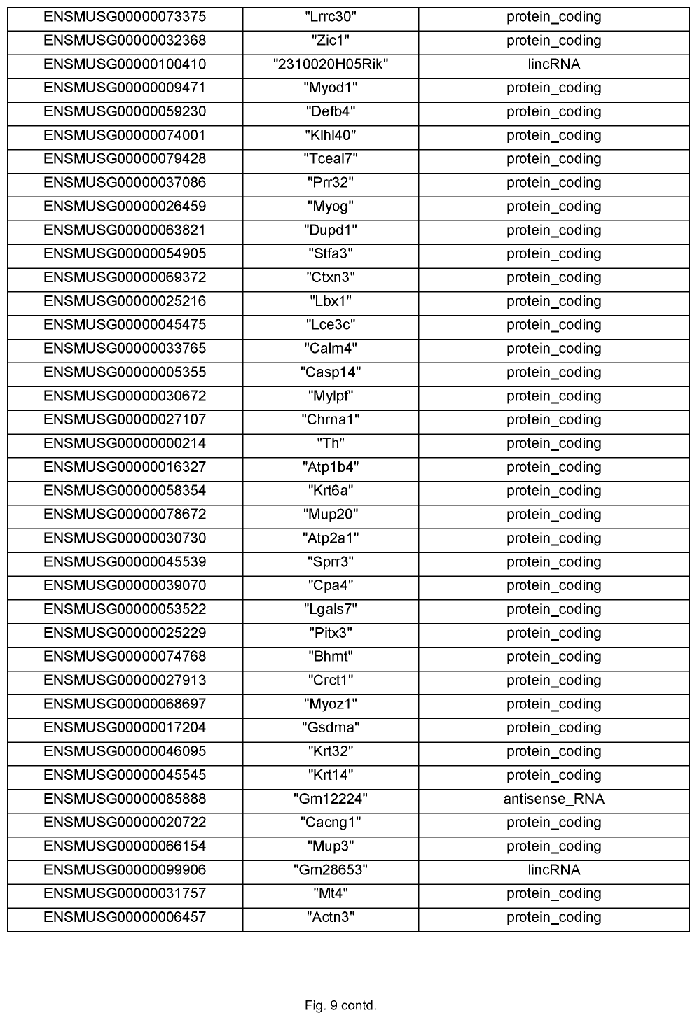

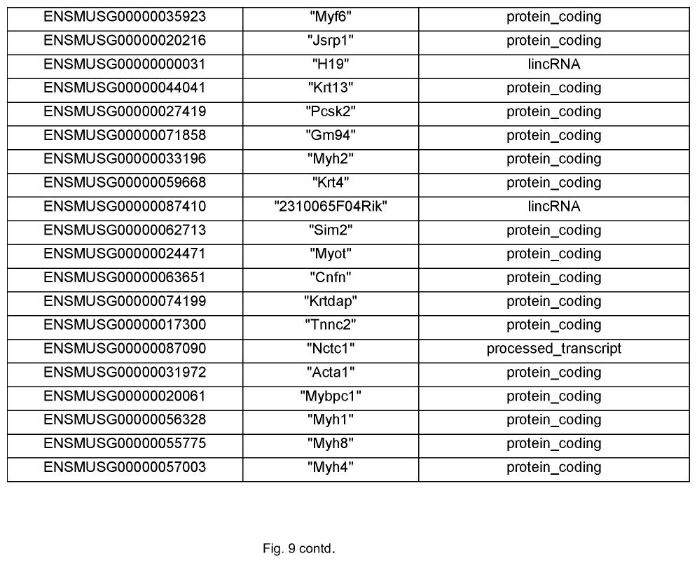

Transcriptomics changes in the sarcoidosis mice model was examined. RNA was extracted from the lungs of mice challenged with microparticles (sarcoidosis model) and injected with saline daily or treated with daily intraperitoneal injection of MIA-602 (5 μg), or extracted from a control group that was never challenged and did not receive MIA-602. RNA was isolated from lung tissue over the course of three weeks, and RNASeq was performed. 778 genes were upregulated, and 293 genes were downregulated (508 protein coding, or TEC genes and rest noncoding) in mice with granuloma that were treated with daily MIA-602 compared to those with granuloma that were treated with saline. Genes that exhibited more than 2.5-fold differential expression upon treatment with MIA-602 are provided as FIG. 9. In various aspects, the disclosure provides a method of characterizing a subject's response to sarcoidosis treatment, wherein the method comprises measuring the expression of one or more of the genes set forth in FIG. 9. In various aspects, a 2.5-fold differential expression compared to the level of expression prior to treatment indicates a therapeutic response to treatment (i.e., the disorder is treated, one more symptoms of the disorder are alleviated, etc.).

The entire document is intended to be related as a unified disclosure, and it should be understood that all combinations of features described herein are contemplated, even if the combination of features is not found together in the same sentence, or paragraph, or section of this document. In addition, the invention includes, as an additional aspect, all embodiments of the invention narrower in scope in any way than the variations specifically mentioned above. With respect to aspects of the invention described or claimed with “a” or “an,” it should be understood that these terms mean “one or more” unless context unambiguously requires a more restricted meaning. With respect to elements described as one or more within a set, it should be understood that all combinations within the set are contemplated. If aspects of the invention are described as “comprising” a feature, embodiments also are contemplated “consisting of” or “consisting essentially of” the feature.

Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention. Additional features and variations of the invention will be apparent to those skilled in the art from the entirety of this application, and all such features are intended as aspects of the invention.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of reducing lung inflammation in a mammalian subject having granulomatous lung disease, the method comprising administering a GHRH antagonist to the mammalian subject.

2. The method of claim 1, wherein the GHRH antagonist comprises the amino acid sequence (Formula I/SEQ ID NO: 2): R¹-Tyr¹-D-Arg²-Asp³-A⁴-Ile⁵-A⁶-Thr⁷-A⁸-Har⁹-A¹⁰-A¹¹-A¹²-Val¹³-Leu¹⁴-A¹⁵- Gln¹⁶-A¹⁷-Ser¹⁸-Ala¹⁹-A²⁰-A²¹-Leu²²-Leu²³-Gln²⁴-Asp²⁵-Ile²⁶-Nle²⁷-D-Arg²⁸-A²⁹-R²-R³-NH₂,
wherein R¹ is PhAc (phenylacetyl), Nac (naphthylacetyl), Oct (octanoyl), N-Me-Aib (N-methyl-alpha-aminoisobutyroyl), Dca (dichloroacetyl), Ac-Ada (acetyl-12-aminododecanoyl), Fer (ferulyl), Ac-Amc (acetyl-8-aminocaprylyl), Me-NH-Sub (methyl-NH-suberyl), PhAc-Ada (phenylacetyl 12-aminododecanoyl), Ac-Ada-D-Phe, Ac-Ada-Phe, Dca-Ada(dichloroacetyl-12-aminododecanoyl), Nac-Ada, Ada-Ada, or CH₃(CH₂)₁₀—CO-Ada;
A⁴ is Ala or Me-Ala;
A⁶ is Cpa (para-chlorophenylalanine) or Phe(F)₅;
A⁸ is Ala, Pal (pyridylalanine), Dip ((3,3-diphenyl)alanine), or Me-Ala;
A¹⁰ is Fpa5 or Tyr(Alk) where Alk is Me or Et;
A¹¹ is His or Arg; A¹² is Lys, a string of 2 to 11 lysine residues, Lys(Me)₂, or Orn (ornithine);
A¹⁵ is Abu (alpha-aminobutyric acid) or Orn;
A¹⁷ is Leu or Glu;
A²⁰ is Har (homoarginine) or His;
A²¹ is Lys, Lys(Me)₂ or Orn;
A²⁹ is Har, Arg or Agm (agmatine);
R₂ is β-Ala, Amc (8-aminocaprylyl), Apa (5-aminopentanoyl), Ada (12-aminododecanoyl), AE₂A (8-amino-3,6-dioxaoctanoyl), AE₄P (15-amino-4,7,10,13-tetraoxapentadecanoyl), ε-Lys(α-NH₂) (a Lys residue, the 8-amino group of which is acylated by the carbonyl group of an N-terminally located amino acid; the α-amino group of the Lys residue is free), Agm (agmatine), or absent; and
R³ is Lys(Oct), Ahx (6-aminohexanoyl), or absent.

3. The method of claim 1, wherein the GHRH antagonist is MIA-602, MIA-604, MIA-606, MIA-610, MIA-640, or MIA-690.

4. The method of claim 1, wherein the GHRH antagonist is MIA-602.

5. The method of claim 1, wherein the GHRH antagonist is administered via intradermal, intramuscular, intraperitoneal, intravenous, intraarterial, subcutaneous, epidural, sublingual, intranasal, intracerebral, intraventricular, intrathecal, intravaginal, transdermal, rectally, inhalation, intrapulmonary, intra-airway, intrabronchial, intratracheal, or topical delivery.

6. The method of claim 5, wherein the GHRH antagonist is administered via intranasal, inhalation, intrapulmonary, intra-airway, intrabronchial, or intratracheal delivery.

7. The method of claim 3, wherein the GHRH antagonist is administered via intradermal, intramuscular, intraperitoneal, intravenous, intraarterial, subcutaneous, epidural, sublingual, intranasal, intracerebral, intraventricular, intrathecal, intravaginal, transdermal, rectally, inhalation, intrapulmonary, intra-airway, intrabronchial, intratracheal, or topical delivery.

8. The method of claim 7, wherein the GHRH antagonist is administered via intranasal, inhalation, intrapulmonary, intra-airway, intrabronchial, or intratracheal delivery.

9. The method of claim 4, wherein the GHRH antagonist is administered via intradermal, intramuscular, intraperitoneal, intravenous, intraarterial, subcutaneous, epidural, sublingual, intranasal, intracerebral, intraventricular, intrathecal, intravaginal, transdermal, rectally, inhalation, intrapulmonary, intra-airway, intrabronchial, intratracheal, or topical delivery.

10. The method of claim 9, wherein the GHRH antagonist is administered via intranasal, inhalation, intrapulmonary, intra-airway, intrabronchial, or intratracheal delivery.


SEQUENCE LISTING (9 sequences)

SEQ ID NO: 1 - protein, 29 aa, Homo sapiens
  REGION 1..29: note = MISC_FEATURE - hGHRH (1-29)HN2
  SITE 29: note = Amidated residue
YADAIFTNSYRKVLGQLSARKLLQDIMSR

SEQ ID NO: 2 - protein, 43 aa, synthetic construct
  REGION 1..43: note = Synthetic Polypeptide
  VARIANT 1..2: note = MISC_FEATURE - This region may encompass one of the following: phenylacetyl (PhAc), acetyl-12-aminododecanoyl (Nac), octanoyl (Oct), N-methyl-alpha-aminoisobutyroyl (N-Me-Aib), dichloroacetyl (Dca), acetyl-12-aminododecanoyl (Ac-Ada), ferulyl (Fer), acetyl-8-aminocaprylyl (Ac-Amc), methyl-NH-suberyl (Me-NH-Sub), phenylacetyl 12-aminododecanoyl (PhAc-Ada), acetyl-12-aminododecanoyl-D-Phe, acetyl-12-aminododecanoyl-Phe, Dca-Ada(dichloroacetyl-12-aminododecanoyl), naphthylacetyl (Nac), acetyl-12-aminododecanoyl-12-aminododecanoyl, 12-aminododecanoyl-12-aminododecanoyl, or CH3(CH2)10-CO-12-aminododecanoyl
  SITE 4: note = MISC_FEATURE - D-Arg
  VARIANT 6: note = MISC_FEATURE - Xaa is Ala or Methyl-Ala
  VARIANT 8: note = MISC_FEATURE - Xaa is para-chlorophenylalanine (Cpa) or pentafluoro-phenylalanine (Phe(F)5)
  VARIANT 10: note = MISC_FEATURE - Xaa is Ala, Pal (pyridylalanine), Dip ((3,3-diphenyl)alanine), or Methyl-Ala
  SITE 11: note = MISC_FEATURE - Xaa is Homoarginine (Har)
  VARIANT 12: note = MISC_FEATURE - Xaa is pentafluoro-phenylalanine (FPa5), or Tyr(Alk) where Alk is Methyl or Ethyl
  VARIANT 13: note = MISC_FEATURE - Xaa is His or Arg
  VARIANT 14..24: note = MISC_FEATURE - This region may encompass one of the following: Lys, Lys(0-11), dimethyl-Lys, or ornithine (Orn)
  VARIANT 14..24: note = This region may encompass 0-11 residues
  VARIANT 27: note = MISC_FEATURE - Xaa is alpha-aminobutyric acid (Abu) or ornithine (Orn)
  VARIANT 29: note = MISC_FEATURE - Xaa is Leu or Glu
  VARIANT 32: note = MISC_FEATURE - Xaa is Homoarginine (Har) or His
  VARIANT 33: note = MISC_FEATURE - Xaa is Lys, Dimethyl-Lys (Lys(Me)2) or ornithine (Orn)
  SITE 39: note = MISC_FEATURE - Xaa is norleucine (Nle)
  SITE 40: note = MISC_FEATURE - D-Arg
  VARIANT 41: note = MISC_FEATURE - Xaa is Homoarginine (Har), Arg or agmatine (Agm)
  VARIANT 42: note = MISC_FEATURE - Xaa is Beta-alanine, 8-aminocaprylyl (Amc), 5-aminopentanoyl (Apa), 12-aminododecanoyl (Ada), 8-amino-3,6-dioxaoctanoyl (AE2A), 15-amino-4,7,10,13-tetraoxapentadecanoyl (AE4P), a Lys residue, the 8-amino group of which is acylated by the carbonyl group of an N-terminally located amino acid; the alpha-amino group of the Lys residue is free (E-Lys(alpha-NH2)), agmatine (Agm), or absent
  VARIANT 43: note = MISC_FEATURE - Xaa is Lys-Octanoyl, 6-aminohexanoyl (Ahx), or absent
  SITE 43: note = AMIDATION
XXYRDXIXTXXXXXXXXXXXXXXXVLXQXSAXXLLQDIXRXXX

SEQ ID NO: 3 - protein, 31 aa, synthetic construct
  REGION 1..31: note = Synthetic Polypeptide
  SITE 1: note = MISC_FEATURE - Xaa is Phenylacetyl modified 12-aminododecanoyl (Ada)
  SITE 3: note = MISC_FEATURE - D-Arg
  SITE 7: note = MISC_FEATURE - Xaa is pentafluoro-phenylalanine (Phe(F)5)
  SITE 10: note = MISC_FEATURE - Xaa is Homoarginine (Har)
  SITE 11: note = MISC_FEATURE - Xaa is Methyl-Tyr (Tyr(Me))
  SITE 13: note = MISC_FEATURE - Xaa is ornithine (Orn)
  SITE 16: note = MISC_FEATURE - Xaa is alpha-aminobutyric acid (Abu)
  SITE 22: note = MISC_FEATURE - Xaa is ornithine (Orn)
  SITE 28: note = MISC_FEATURE - Xaa is norleucine (Nle)
  SITE 29: note = MISC_FEATURE - D-Arg
  SITE 30: note = MISC_FEATURE - Xaa is Homoarginine (Har)
  SITE 31: note = MISC_FEATURE - Xaa is agmatine (Agm)
  SITE 31: note = AMIDATION
XYRDAIXTAXXHXVLXQLSAHXLLQDIXRXX

SEQ ID NO: 4 - protein, 31 aa, synthetic construct
  REGION 1..31: note = Synthetic Polypeptide
  SITE 1: note = MISC_FEATURE - Xaa is Phenylacetyl modified 12-aminododecanoyl (Ada)
  SITE 3: note = MISC_FEATURE - D-Arg
  SITE 7: note = MISC_FEATURE - Xaa is pentafluoro-phenylalanine (Phe(F)5)
  SITE 8: note = MISC_FEATURE - Xaa is Methyl-Thr (Thr(Me))
  SITE 10: note = MISC_FEATURE - Xaa is Homoarginine (Har)
  SITE 11: note = MISC_FEATURE - Xaa is Methyl-Tyr (Tyr(Me))
  SITE 13: note = MISC_FEATURE - Xaa is ornithine (Orn)
  SITE 16: note = MISC_FEATURE - Xaa is alpha-aminobutyric acid (Abu)
  SITE 22: note = MISC_FEATURE - Xaa is ornithine (Orn)
  SITE 28: note = MISC_FEATURE - Xaa is norleucine (Nle)
  SITE 29: note = MISC_FEATURE - D-Arg
  SITE 30: note = MISC_FEATURE - Xaa is Homoarginine (Har)
  SITE 31: note = MISC_FEATURE - Xaa is agmatine (Agm)
  SITE 31: note = AMIDATION
XYRDAIXXAXXHXVLXQLSAHXLLQDIXRXX

SEQ ID NO: 5 - protein, 30 aa, synthetic construct
  REGION 1..30: note = Synthetic Polypeptide
  SITE 1: note = MISC_FEATURE - Phenylacetyl modified residue
  SITE 2: note = MISC_FEATURE - D-Ala
  SITE 6: note = MISC_FEATURE - Xaa is para-chlorophenylalanine (Cpa)
  SITE 9: note = MISC_FEATURE - Xaa is Homoarginine (Har)
  SITE 10: note = MISC_FEATURE - Xaa is pentafluoro-phenylalanine (Fpa)
  SITE 12: note = MISC_FEATURE - Xaa is ornithine (Orn)
  SITE 15: note = MISC_FEATURE - Xaa is alpha-aminobutyric acid (Abu)
  SITE 21: note = MISC_FEATURE - Xaa is ornithine (Orn)
  SITE 27: note = MISC_FEATURE - Xaa is norleucine (Nle)
  SITE 28: note = MISC_FEATURE - D-Arg
  SITE 29: note = MISC_FEATURE - Xaa is Homoarginine (Har)
  SITE 30: note = MISC_FEATURE - Xaa is 12-aminododecanoyl (Ada)
  SITE 30: note = AMIDATION
YADAIXTAXXHXVLXQLSAHXLLQDIXRXX

SEQ ID NO: 6 - protein, 31 aa, synthetic construct
  REGION 1..31: note = Synthetic Polypeptide
  SITE 1: note = MISC_FEATURE - Xaa is Phenylacetyl modified 12-aminododecanoyl (Ada)
  SITE 3: note = MISC_FEATURE - D-Arg
  SITE 7: note = MISC_FEATURE - Xaa is para-chlorophenylalanine (Cpa)
  SITE 10: note = MISC_FEATURE - Xaa is Homoarginine (Har)
  SITE 11: note = MISC_FEATURE - Xaa is pentafluoro-phenylalanine (Fpa)
  SITE 13: note = MISC_FEATURE - Xaa is ornithine (Orn)
  SITE 16: note = MISC_FEATURE - Xaa is alpha-aminobutyric acid (Abu)
  SITE 22: note = MISC_FEATURE - Xaa is ornithine (Orn)
  SITE 28: note = MISC_FEATURE - Xaa is norleucine (Nle)
  SITE 29: note = MISC_FEATURE - D-Arg
  SITE 30: note = MISC_FEATURE - Xaa is Homoarginine (Har)
  SITE 31: note = MISC_FEATURE - Xaa is 12-aminododecanoyl (Ada)
  SITE 31: note = MISC_FEATURE - C-Term NH2
  SITE 31: note = AMIDATION
XYRDAIXTAXXHXVLXQESAHXLLQDIXRXX

SEQ ID NO: 7 - protein, 30 aa, synthetic construct
  REGION 1..30: note = Synthetic Polypeptide
  SITE 1: note = MISC_FEATURE - Xaa is Phenylacetyl modified 12-aminododecanoyl (Ada)
  SITE 3: note = MISC_FEATURE - D-Arg
  SITE 7: note = MISC_FEATURE - Xaa is para-chlorophenylalanine (Cpa)
  SITE 10: note = MISC_FEATURE - Xaa is Homoarginine (Har)
  SITE 11: note = MISC_FEATURE - Xaa is pentafluoro-phenylalanine (Fpa)
  SITE 13: note = MISC_FEATURE - Xaa is ornithine (Orn)
  SITE 16: note = MISC_FEATURE - Xaa is alpha-aminobutyric acid (Abu)
  SITE 22: note = MISC_FEATURE - Xaa is ornithine (Orn)
  SITE 28: note = MISC_FEATURE - Xaa is norleucine (Nle)
  SITE 29: note = MISC_FEATURE - D-Arg
  SITE 30: note = MISC_FEATURE - Xaa is Homoarginine (Har)
  MOD_RES 30: note = AMIDATION
XYRDAIXTAXXHXVLXQLSAHXLLQDIXRX

SEQ ID NO: 8 - protein, 30 aa, synthetic construct
  REGION 1..30: note = Synthetic Polypeptide
  SITE 1: note = MISC_FEATURE - Xaa is Phenylacetyl modified 12-aminododecanoyl (Ada)
  SITE 3: note = MISC_FEATURE - D-Arg
  SITE 7: note = MISC_FEATURE - Xaa is pentafluoro-phenylalanine (Fpa)
  SITE 10: note = MISC_FEATURE - Xaa is Homoarginine (Har)
  SITE 11: note = MISC_FEATURE - Xaa is Methyl-Tyr (Tyr(Me))
  SITE 13: note = MISC_FEATURE - Xaa is ornithine (Orn)
  SITE 16: note = MISC_FEATURE - Xaa is alpha-aminobutyric acid (Abu)
  SITE 21: note = MISC_FEATURE - Xaa is ornithine (Orn)
  SITE 28: note = MISC_FEATURE - Xaa is norleucine (Nle)
  SITE 29: note = MISC_FEATURE - D-Arg
  SITE 30: note = MISC_FEATURE - Xaa is Homoarginine (Har)
XYRDAIXTAXXHXVLXQLSAXKLLQDIXRX

SEQ ID NO: 9 - protein, 11 aa, synthetic construct
  VARIANT 1..11: note = This sequence may encompass 0-11 residues
KKKKKKKKKKK